(19) United States
(12) Patent Application Publication
(10) Pub. No.: US 2025/0073323 A1
(43) Pub. Date: Mar. 6, 2025

(54) PROCESS FOR PRODUCING PERSONALIZED CANCER IMMUNOTHERAPY

(71) Applicant: ANYADI, LLC, Wilmington, DE (US)

(72) Inventors: Marc Andrew GILLIG, Newington, CT (US); Rachit OHRI, Ellicott City, MD (US)

(21) Appl. No.: 18/293,994

(22) PCT Filed: Aug. 8, 2022
(86) PCT No.: PCT/US2022/039694

§ 371 Date: Jan. 31, 2024

Related U.S. Application Data
(60) Provisional application No. 63/230,161, filed on Aug. 6, 2021.

(51) Int. Cl. A61K 39/00; C07K 16/28; C07K 16/30

(52) U.S. Cl. CPC A61K 39/0011; A61K 40/15; C07K 16/2833; C07K 16/30; C07K 2317/31; C07K 2317/732

(57) ABSTRACT

The invention of the current disclosure includes methods and compositions useful for producing personalized cancer immunotherapies which target tumor-associated neoepitopes. Also included are methods for treating cancer in subjects in need thereof.

### CROSS REFERENCE TO RELATED APPLICATION

The present application is entitled to priority under 35 U.S.C. § 119(e) to U.S. Provisional Patent Application No. 63/230,161, filed Aug. 6, 2021, which is hereby incorporated by reference in its entirety herein.

### FIELD OF THE INVENTION

The invention relates generally to a process for producing personalized cancer immunotherapies which may be customized on a patient-by-patient basis. More specifically, it relates to antibodies targeting neoepitopes on the surface of diseased cells, with the antibodies acting in concert with immune cells.

### BACKGROUND OF THE INVENTION

Cancer immunotherapy is the use of immune cells, immune system-related molecules, or immunization methods to treat cancer. Examples of immunotherapy approaches include checkpoint blockade, targeted therapy with monoclonal antibodies, adoptive cellular therapies, and therapeutic immunization with cancer antigens. Checkpoint blockade antibodies (e.g. ipilimumab, pembrolizumab) are intended to boost or rejuvenate natural anticancer immune responses, while targeted antibody therapies are intended to direct cytotoxic responses toward cancer cells overexpressing certain surface molecules (e.g. cetuximab specific for Epidermal Growth Factor Receptor, EGFR). Cellular therapies include ex vivo expansion and administration of autologous or allogeneic immune cells (e.g. T cells, natural killer (NK) cells), as well as engineering cells to express chimeric antigen receptors (CARs) specific for cell surface molecules such as CD19. In the related field of therapeutic immunization, research is ongoing to develop strategies based on the “neoantigens” that arise from mutations, which are often presented (as “neoepitope” peptides) by HLA molecules on the surface of cancer cells.

Ideal cancer treatments should specifically destroy only cancerous cells while leaving normal cells unaffected, resulting in high efficacy and low or absent toxicity. Unfortunately, this remains an unrealized vision. Checkpoint blockade, due to its non-specific activation of immune cells, often results in adverse events. Most CAR-T cell therapies target both cancerous cells and normal cells, based on the most commonly chosen target antigens, and therefore lack true cancer specificity. While the theoretical benefit of neoepitope immunization is its true cancer specificity, significant clinical success has yet to be achieved with this approach. Broadly speaking, immunotherapy approaches thus far have suffered either from lack of specificity, high toxicity, lack of efficacy, challenges in generation, or a combination of the above.

Therefore, there is a need in the art for efficacious cancer-specific therapies that do not affect normal cells and cause minimal adverse events, if any. The current invention addresses this need.

### SUMMARY OF THE INVENTION

The following presents a simplified summary of aspects of the invention in order to provide a basic understanding of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

In certain embodiments of the invention, a composition for treating cancer is provided, comprising one or more antibodies (or antigen-binding fragments thereof) that specifically bind a tumor-specific epitope-HLA complex, and an effective amount of a cytotoxic immune effector cell, wherein the composition is personalized given that [a] the tumor-specific epitope is a patient-specific neoepitope displayed on the HLA (Human Leukocyte Antigen) system, and [b] the cytotoxic immune effector cells are capable of engaging the antibody (or antibodies, or antibody fragments) through their Fc-gamma (Fcγ) receptors on the cell surface, and subsequently inducing ADCC (Antibody Dependent Cell-mediated Cytotoxicity) or ADCP (Antibody Dependent Cell-mediated Phagocytosis), being a cell type selected from the group consisting of NK cells, macrophages, monocytes, neutrophils, dendritic cells, or any combination thereof.

In certain embodiments, a method of producing a composition for treating cancer is provided, comprising the following steps: obtaining a tumor specimen from a subject and identifying tumor-specific genetic mutations expressed by the tumor through sequencing, generating HLA/neoepitope complexes based on the identified tumor-specific genetic mutations, immunizing a non-human animal with the HLA/neoepitope complexes, isolating B cells that produce neoepitope-specific antibodies or antigen-binding fragments thereof from the immunized non-human animal, verifying that the neoepitope-specific antibodies bind to the tumor, producing an effective amount of the neoepitope-specific antibodies or antibody fragments, and combining [a] an effective amount of the antibody (or antibodies or antibody fragments) that specifically bind the tumor-specific epitope-HLA complex, along with [b] an effective amount of a cytotoxic immune effector cell capable of engaging the antibody (or antibodies, or antibody fragments) through the Fc-gamma (Fcγ) receptors on the cell surface, and subsequently inducing ADCC (Antibody Dependent Cell-mediated Cytotoxicity) or ADCP (Antibody Dependent Cell-mediated Phagocytosis), being selected from the group consisting of NK cells, macrophages, monocytes, neutrophils, dendritic cells, or any combination thereof.

In one aspect, the invention includes a composition for treating cancer, said composition comprising:
a. one or more antibodies, or antigen-binding fragments thereof that specifically bind one or more tumor-specific epitopes, and
b. an effective amount of a cytotoxic immune effector cell.

In certain embodiments, the composition is personalized.

In certain embodiments, the tumor-specific epitope is a neoepitope.

In certain embodiments, the antibodies or antigen-binding fragments thereof are humanized.

In certain embodiments, the antibodies or antigen-binding fragments thereof are fully human.

In certain embodiments, the antibodies or antigen-binding fragments thereof are afucosylated.

In certain embodiments, the antibody or antigen-binding fragment thereof is bispecific.

In certain embodiments, the tumor-specific epitope is a neoepitope/HLA complex.

In certain embodiments, the cytotoxic immune effector cells are a cell type selected from the group consisting of NK cells, macrophages, monocytes, neutrophils, dendritic cells, and any combination thereof.

In certain embodiments, the cytotoxic immune effector cells are irradiated.

In another aspect, the invention includes a method of producing a composition for treating cancer, said method comprising:
a. obtaining a tumor specimen from a subject and identifying tumor-specific genetic mutations expressed by the tumor through sequencing,
b. generating HLA/neoepitope complexes based on the identified tumor-specific genetic mutations,
c. immunizing a non-human animal with the HLA/neoepitope complexes,
d. isolating B cells from the immunized non-human animal that produce HLA/neoepitope-specific antibodies or facilitate the generation of antigen-binding fragments thereof,
e. verifying that the HLA/neoepitope-specific antibodies bind to the tumor,
f. producing an effective amount of the HLA/neoepitope-specific antibodies, and
g. combining the effective amount of the HLA/neoepitope-specific antibodies with an effective amount of a cytotoxic immune effector cell.

In certain embodiments, the sequencing is next-generation sequencing.

In certain embodiments, the non-human animal is humanized.

In certain embodiments, the non-human animal produces fully-human antibodies.

In certain embodiments, the non-human humanized animal is selected from the group consisting of a rat, a mouse, a rabbit, a donkey, a goat, a camel, a llama, an alpaca, and a shark.

In certain embodiments, the HLA/neoepitope-specific antibodies are generated through a non-animal method.

In certain embodiments, the non-animal method is phage display.

In certain embodiments, the antibodies or antigen-binding fragments thereof are afucosylated.

In certain embodiments, the isotype of the antibodies supports ADCC or ADCP.

In certain embodiments, the isotype of the antibodies are further modified from an isotype that does not support ADCC or ADCP to an isotype that supports ADCC or ADCP.

In certain embodiments, HLA/neoepitope complexes are selected from the group consisting of monomeric complexes, pentameric complexes, tetrameric complexes, dextran-linked complexes, and any combination thereof.

In certain embodiments, the cytotoxic immune effector cells are a cell type selected from the group consisting of NK cells, macrophages, monocytes, neutrophils, dendritic cells, and any combination thereof.

In another aspect, the invention includes a method of producing a composition for treating cancer, said method comprising:
a. obtaining a tumor specimen from a subject and identifying tumor-specific genetic mutations expressed by the tumor through sequencing,
b. generating HLA/neoepitope complexes based on the identified tumor-specific genetic mutations,
c. immunizing a non-human animal with the HLA/neoepitope complexes,
d. isolating B cells from the immunized non-human animal that produce HLA/neoepitope-specific antibodies or facilitate the generation of antigen-binding fragments thereof,
e. verifying that the HLA/neoepitope-specific antibodies bind to the tumor,
f. generating one or more a chimeric antigen receptors (CARs) comprising the antigen binding domains from the HLA/neoepitope-specific antibodies, and
g. expressing the CARs in a cytotoxic immune effector cell.

In certain embodiments, the sequencing is next-generation sequencing.

In certain embodiments, the non-human animal is humanized.

In certain embodiments, the non-human animal produces fully-human antibodies.

In certain embodiments, the non-human humanized animal is selected from the group consisting of a rat, a mouse, a rabbit, a donkey, a goat, a camel, a llama, an alpaca, and a shark.

In certain embodiments, the HLA/neoepitope-specific antibodies are generated through a non-animal method.

In certain embodiments, the non-animal method is phage display.

In certain embodiments, generating the CAR further comprises the step of generating an scFv based on the antigen-binding domains of the antibodies.

In certain embodiments, the CAR further comprises a transmembrane domain, a signaling domain, and a costimulatory domain.

In certain embodiments, the HLA/neoepitope complexes are selected from the group consisting of monomeric complexes, pentameric complexes, tetrameric complexes, dextran-linked complexes, and any combination thereof.

In certain embodiments, the cytotoxic immune effector cells are a cell type selected from the group consisting of NK cells, T cells, NK T cells, or any combination thereof.

In certain embodiments, the cytotoxic immune effector cell is an NK cell.

In certain embodiments, the cytotoxic immune effector cell is a CD8+ T cell.

In another aspect, the invention includes a method of treating cancer in a subject in need thereof, said method comprising:
a. obtaining a tumor specimen from the subject and identifying tumor-specific neoepitopes expressed by the tumor through sequencing,
b. generating HLA/neoepitope complexes from the identified tumor-specific neoepitopes,
c. immunizing a non-human mammal with the HLA/neoepitope complexes,
d. isolating B cells that produce HLA/neoepitope-specific antibodies or antigen-binding fragments thereof from the immunized non-human mammal,
e. verifying that the HLA/neoepitope-specific antibodies bind to the tumor,
f. producing an effective amount of the HLA/neoepitope-specific antibodies, and
g. administering an effective amount of the HLA/neoepitope-specific antibodies and an effective amount of a cytotoxic immune effector cell to the subject, thereby treating the cancer.

In certain embodiments, the sequencing is next-generation sequencing.

In certain embodiments, the non-human animal is humanized.

In certain embodiments, the non-human animal produces fully-human antibodies.

In certain embodiments, the non-human humanized animal is selected from the group consisting of a rat, a mouse, a rabbit, a donkey, a goat, a camel, a llama, an alpaca, and a shark.

In certain embodiments, the HLA/neoepitope-specific antibodies are generated through a non-animal method.

In certain embodiments, the non-animal method is phage display.

In certain embodiments, the HLA/neoepitope-specific antibodies are afucosylated.

In certain embodiments, the isotype of the antibodies supports ADCC or ADCP.

In certain embodiments, the isotype of the antibodies are further modified from an isotype that does not support ADCC or ADCP to an isotype that supports ADCC or ADCP.

In certain embodiments, the HLA/neoepitope complexes are selected from the group consisting of monomeric complexes, pentameric complexes, tetrameric complexes, dextran-linked complexes, and any combination thereof.

In certain embodiments, the cytotoxic immune effector cells are a cell type selected from the group consisting of NK cells, macrophages, monocytes, neutrophils, dendritic cells, or any combination thereof.

In another aspect, the invention includes a method of treating cancer in a subject in need thereof, said method comprising:
a. obtaining a tumor specimen from the subject and identifying tumor-specific neoepitopes expressed by the tumor through sequencing,
b. generating HLA/neoepitope complexes from the identified tumor-specific neoepitopes,
c. immunizing a non-human mammal with the HLA/neoepitope complexes,
d. isolating B cells that produce HLA/neoepitope-specific antibodies or antigen-binding fragments thereof from the immunized non-human mammal,
e. verifying that the HLA/neoepitope-specific antibodies bind to the tumor,
f. generating a chimeric antigen receptor (CAR) comprising the antigen-binding domains from the HLA/neoepitope-specific antibodies,
g. expressing the CAR in a cytotoxic immune effector cell, and
h. administering to the subject an effective amount of the cytotoxic immune effector cell, thereby treating the cancer.

In certain embodiments, the sequencing is next-generation sequencing.

In certain embodiments, the non-human animal is humanized.

In certain embodiments, the non-human humanized animal is selected from the group consisting of a rat, a mouse, a rabbit, a donkey, a goat, a camel, a llama, an alpaca, and a shark.

In certain embodiments, the HLA/neoepitope-specific antibodies are generated through a non-animal method.

In certain embodiments, the non-animal method is phage display.

In certain embodiments, the HLA/neoepitope complexes are selected from the group consisting of monomeric complexes, pentameric complexes, tetrameric complexes, dextran-linked complexes, and any combination thereof.

In certain embodiments, generating the CAR further comprises the step of generating an scFv based on the antigen-binding domains of the antibodies.

In certain embodiments, the CAR further comprises a transmembrane domain, a signaling domain, and a costimulatory domain.

In certain embodiments, the HLA/neoepitope complexes are selected from the group consisting of monomeric complexes, pentameric complexes, tetrameric complexes, dextran-linked complexes, and any combination thereof.

In certain embodiments, the cytotoxic immune effector cells are a cell type selected from the group consisting of NK cells, T cells, NK T cells, or any combination thereof.

In certain embodiments, the cytotoxic immune effector cell is an NK cell.

In certain embodiments, the cytotoxic immune effector cell is a CD8+ T cell.

### BRIEF DESCRIPTION OF THE FIGURES

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 is a simplified flow chart of the process by which the proposed personalized cancer immunotherapy is manufactured.

FIG. 2 is a mechanism of action of the present invention. Implicit in FIG. 2 are scenarios where the CD16 on the surface of the effector immune cells is instead any Fc-gamma receptor, including but not limited to CD16, or CD32, or CD64. FIG. 2 legends correspond as follows: 201 Mutations result in translation of non-self amino acid sequences, 202 The antigen processing pathway leads to the presentation of neoepitopes on HLA molecules, 203 Personalized TCR-like antibodies against the neoepitope-HLA complex bind to cancer cells with high specificity, 204 CD16 FcγRIII) on effector cells binds with high affinity to the Fc region, activating the cytotoxic effector function, 205 cytotoxic activity leads to specific killing of cancer cells.

FIG. 3 is a simplified flow chart of the process by which the proposed personalized cancer immunotherapy is manufactured, in the preferred embodiment.

FIG. 4 is a mechanism of action of the proposed immunotherapy, in the preferred embodiment. FIG. 4 legends correspond as follows: 401 Frameshift mutations result in translation of non-self amino acid sequences, 402 The antigen processing pathway leads to the presentation of neoepitopes on HLA molecules, 403 Personalized TCR-like antibodies against the neoepitope-HLA complex bind to cancer cells with high specificity, 404 CD16 (FcγRIII) on NK cells binds with high affinity to the afucosylated Fc region, activating the cytotoxic effector function, 405 Degranulation of cytotoxic granules, and/or secretion of cytokines, leads to specific killing of cancer cells.

FIG. 5 is a detailed flow chart of the process by which the proposed personalized cancer immunotherapy is manufactured and administered, in the preferred embodiment. FIG. 5 legends correspond as follows: 502 Blood Sample, 504 Tumor sample, 506 Next-generation DNA sequencing, 508 RNA sequencing, 510 Identify expressed frameshift mutations in tumor, 512 Predict frameshift-derived high-affinity neoepitopes based on HLA haplotype, 514 Produce HLA neoepitope tetramers, 516 Immunize OmniRats with HLA-neoepitope tetramers, 518 Immunize OmniRats with HLA-neoepitope tetramers a second time, 520 Enrich B cells from spleen and lymph nodes, 522 Stain B cells with HLA-neoepitope tetramers, 524 Single cell sorting of neoepitope-specific B cells, 526 Test Ab:Ag by ELISA524, 528 RT-PCR and sequencing of positives, 530 Clone (human) variable region sequences into human antibody vectors, 532 Produce small test-batches of candidate anti-neoepitope antibodies using producer cell lines, 534 Create single-cell suspension of patient's tumor cells, 536 Cryopreservation, 538 Thaw patient's tumor cells, 540 Stain tumor cells with candidate anti-neoepitope antibodies by ELISA, 542 Culture an NK cell line (that expresses CD16) in cytotoxicity-enhancing media, 544 Larger-scale GMP production of successful antibodies, using producer cells engineered to produce afucosylated antibodies, 546 Successful tumor-binding HLA-neoepitope-specific antibodies are selected, 548 Irradiate NK cells, 550 NK cell infusion, and 552 Antibody infusion.

FIG. 6 is a detailed flowchart of an embodiment where SNV-derived neoepitopes are targeted. FIG. 6 legends correspond as follows: 601 Identify expressed frameshift mutations in tumor, 602 predict frameshift-derived high-affinity neoepitopes based on HLA haplotype, 603 test Ab:Ag binding by ELISA, 604 stain tumor cells with candidate anti-neoepitope antibodies, 605 successful tumor-binding HLA-neoepitope-specific antibodies are selected.

FIG. 7 is a mechanism of action of an embodiment where SNV-derived neoepitopes are targeted. FIG. 7 legends correspond as follows: 701 SNV mutations result in translation of amino acid sequences containing a single amino acid change, 702 the antigen processing pathway leads to the presentation of neoepitopes on HLA molecules, 703 personalized TCR-like antibodies against the neoepitope-HLA complex bind to cancer cells with high specificity, 704 CD16 (FcγRIII) on NK cells binds with high affinity to the afucosylated Fc region activating the cytotoxic effector function, 705 degranulation of cytotoxic granules, an/or secretion of cytokines leads to specific killing of cancer cells.

FIG. 8 is a detailed flowchart of an embodiment where monomers, or other multimers besides tetramers, of HLA-neoepitope complexes are used for immunization and/or testing.

FIG. 8 legends correspond as follows: 801 Produce HLA-neoepitope monomers or multimers, 802 immunize OmniRats with HLA-neoepitope monomers or multimers, 803 immunize OmniRats with HLA-neoepitope monomers or multimers a second time, 804 stain B cells with HLA-neoepitope monomers or multimers.

FIG. 9 is a detailed flowchart of an embodiment where the TCR-like antibodies are engineered in some way, e.g. bispecificity, or a modified Fc region to optimize effector function.

FIG. 9 legends correspond as follows: 901 engineering antibodies to be bi-specific and/or to have modified Fc region.

FIG. 10 is a mechanism of action of an embodiment where the TCR-like antibodies are engineered in some way, e.g. bispecificity, or a modified Fc region to optimize effector function.

FIG. 9 legends correspond as follows: 1001 Frameshift mutations result in translation of non-self amino acid sequences, 1002 The antigen processing pathway leads to the presentation of neoepitopes on HLA molecules, 1003 Personalized TCR-like and/or bispecific antibodies against the neoepitope-HLA complex bind to cancer cells with high specificity, 1004 CD16 (FcγRIII) on NK cells binds with high affinity to the engineered Fc region, activating the cytotoxic effector function, 1005 Degranulation of cytotoxic granules, and/or secretion of cytokines, leads to specific killing of cancer cells.

FIG. 11 is a detailed flowchart of an embodiment where different cell types are administered to the patient, such as macrophages, neutrophils, monocytes, or dendritic cells. FIG. 11 legends correspond as follows: 1101 culture macrophages, neutrophils, monocytes, or dendritic cells in cytotoxicity-enhancing media, 1102 irradiate cells (only necessary if an immortalized cell line).

FIG. 12 is a mechanism of action of an embodiment where different cell types are administered to the patient, such as macrophages, neutrophils, monocytes, or dendritic cells.

FIG. 12 legends correspond as follows: 1201 Frameshift mutations result in translation of non-self amino acid sequences, 1202 The antigen processing pathway leads to the presentation of neoepitopes on HLA molecules, 1203 Personalized TCR-like antibodies against the neoepitope-HLA complex bind to cancer cells with high specificity, 1204 CD16 (FcγRIII) or CD32 (FcγRII) on effector cells binds with high affinity to the afucosylated Fc region, activating the cytotoxic effector function (ADCC or ADCP), 1205 Antibody-dependent cellular phagocytosis (ADCP) or other cytotoxic mechanism.

FIG. 13 is a detailed flowchart of an embodiment where the cells administered to the patient are genetically engineered. FIG. 13 legends correspond as follows: 1301 genetically engineer cytotoxic effector cells (e.g. knock-out certain genes), 1302 irradiate engineered cells.

FIG. 14 is a mechanism of action of an embodiment where the cells administered to the patient are genetically engineered. FIG. 14 legends correspond as follows: 1401 Frameshift mutations result in translation of non-self amino acid sequences, 1402 The antigen processing pathway leads to the presentation of neoepitopes on HLA molecules, 1403 Personalized TCR-like antibodies against the neoepitope-HLA complex bind to cancer cells with high specificity, 1404 Fcγ receptor on engineered effector cells binds with high affinity to the afucosylated Fc region, activating the cytotoxic effector function, 1405 Degranulation of cytotoxic granules, and/or secretion of cytokines, leads to specific killing of cancer cells.

FIG. 15 is a detailed flowchart of an embodiment where the NK cells administered to the patient are autologous or, alternatively, allogeneic cells either from blood donors or cord blood. FIG. 15 legends correspond as follows: 1501 culture autologous NK cells, or allogeneic NK cells sourced from cord blood or donor blood, in cytotoxicity-enhancing media.

FIG. 16 is a detailed flowchart of an embodiment where the administered cytotoxic effector cells express a CAR construct, and the neoepitope specificity is conferred not by an antibody but by the scFv portion of the CAR. Implicit in FIG. 16 is the option of including the administration of TCR-like antibodies generated against the neoepitope-HLA complexes. FIG. 16 legends correspond as follows: 1601 successful tumor-binding HLA-neoepitope-specific antibodies are converted into scFv sequences and cloned into a CAR construct, 1602 cytotoxic effector cells are transduced with the CAR construct, 1603 infusion of CAR-expressing effector cells.

FIG. 17 is a mechanism of action of an embodiment where the administered cytotoxic effector cells express a CAR construct, and the neoepitope specificity is conferred not by an antibody but by the scFv portion of the CAR. Implicit in FIG. 17 is the option of including the administration of TCR-like antibodies generated against the neoepitope-HLA complexes. FIG. 17 legends correspond as follows: 1701 frameshift mutations result in translation of non-self amino acid sequences, 1702 The antigen processing pathway leads to the presentation of neoepitopes on HLA molecules, 1703 personalized TCR-like single-chain variable fragment (scFv) incorporated into a chimeric antigen receptor (CAR) against the neoepitope-HLA complex bind to cancer cells with high specificity, 1704 cytotoxic effector cells are engineered to express the neoepitope-specific CAR, degranulation of cytotoxic granules, and/or secretion of cytokines, leads to specific killing of cancer cells.

FIG. 18 illustrates the gating strategy for flow cytometry analysis. To assess the cytotoxic activity of NK cells against target cells using flow cytometry, the following gating strategy was used: a large forward scatter/side scatter gate capturing both effector cells and target cells was used to exclude debris. Target (EL4) cells were distinguished from effector (NK) cells based on CFSE staining. Apoptosis and death of target cells were quantified using Annexin V staining of phosphatidyl-serine and SYTOX blue staining of DNA, respectively. Degranulation, i.e. cytotoxic activity, of NK cells was quantified using an antibody against CD107a. Representative dot plots from several different cocultures are shown for demonstration.

FIGS. 19A-19B depicts an ADCC-mediated boost in NK cell killing of target cells via a TCR-like antibody directed against the neoepitope displayed by target cells. EL4 cells were incubated with SIINFEKL peptide to load cell-surface MHC molecules (H2K^{b}) with this specific peptide. FIG. 19A: After 4 hours in monoculture with or without anti-SIINFEKL-H2K^{b} IgG2a antibody, the EL4 cells were stained with SYTOX blue to quantify dead cells. FIG. 19B: represents the same experimental endpoint as in A, but for the co-culture experimental groups. Freshly isolated primary mouse NK cells were added at 3 different Effector:Target ratios, and cocultured with or without antibody for 4 hours. Since the EL4 monocultures demonstrated a statistically significant direct cytotoxic effect of the antibody (without any NK cells), this cytotoxicity contribution (by the antibody alone) was subtracted from the coculture results of the experimental group with the antibody, in order to accurately quantify and compare the sum of [FIG. 19A] the natural cytotoxicity and [FIG. 19B] the ADCC cytotoxicity of NK cells—in the presence or absence of the antibody. The difference between the measured cytotoxicity (along the y-axis) as quantified in the presence of the antibody versus the absence of the antibody represents quantification of the ADCC mechanism per se, for a given E:T ratio. Error bars indicate standard deviation (n=3). Statistics indicate p-values resulting from unpaired t-tests. * p<0.05; ** p<0.01

FIG. 20 illustrates that the release of lactate dehydrogenase (LDH) corroborates dead cell staining quantified by flow cytometry. After the 4-hour incubation, supernatants from the cocultures (and monocultures) with or without antibody described in FIG. 19, were quantified for the presence of LDH using a colorimetric assay of LDH enzymatic activity. Error bars indicate standard deviation (n=3). Statistics indicate p-values resulting from unpaired t-tests. * p<0.05; ** p<0.01.

FIGS. 21A-21B depict ADCC via the TCR-like antibody occurring by apoptosis more than by necrosis, and both ADCC-apoptosis and ADCC-necrosis are boosted by increasing E:T ratios. The data represented in FIG. 19 was further analyzed on the basis of Annexin-V staining to quantify: [FIG. 21A] target cell death through Apoptosis (Annexin-V positive staining) and [FIG. 21B] target-cell death through non-apoptotic Necrosis (Annexin-V negative staining). SIINFEKL peptide-loaded EL4 cells were cultured with or without anti-SIINFEKL-H2K^{b} IgG2a antibody as in the previous figures. In the same experiment, cells were also stained with Annexin-V to quantify cells undergoing apoptosis. By combining SYTOX staining of dead cells with Annexin-V staining of apoptotic cells, it was possible to distinguish between (FIG. 21A) late apoptosis and (FIG. 21B) necrosis, with respect to the increase in target cell death mediated by ADCC. Error bars indicate standard deviation (n=3). Statistics indicate p-values resulting from unpaired t-tests. * p<0.05; ** p<0.01.

FIG. 22 illustrates that Degranulation of NK cells increases in the presence of the antibody as measured by CD107a staining. SIINFEKL peptide-loaded EL4 cells were cultured with or without anti-SIINFEKL-H2K^{b} IgG2a antibody as in the previous figures. In the same experiment, cells were also stained throughout the 4-hour incubation with an antibody against CD107a. CD107a is a marker of degranulation by NK cells, a mechanism used by NK cells to induce target cell killing. NK cells were also cultured alone (NK monocultures) to establish a baseline level of CD107a staining in the absence of any target cells. Error bars indicate standard deviation (n=3). Statistics indicate p-values resulting from unpaired t-tests. *** p<0.001; **** p<0.0001.

FIGS. 23A-23B depict the anti-SIINFEKL-H2K^{b} antibody binding specifically to SIINFEKL-pulsed EL4 cells. In an experiment separate from the one corresponding to FIGS. 18-22, EL4 cells were incubated with SIINFEKL peptide to load cell-surface MHC molecules, or incubated in similar conditions without any peptide. Both peptide-pulsed and unpulsed cells were then incubated with or without the anti-SIINFEKL-H2K^{b} antibody. All four of these experimental groups were subsequently stained with a PE-conjugated secondary antibody specific for mouse IgG—for analysis through flow cytometry. Four replicates were used for each of the four conditions.

FIG. 24 is a diagram illustrating the workflow to generate several novel TCR-like antibodies against a “public” frameshift-derived neoantigen commonly found in AML. Legends correspond as follows: 2401 produce HLA-neoepitope complexes, 2402 immunize OmniRats with HLA-neoepitope complexes, 2403 immunize OmniRats with HLA-neoepitope complexes a second time, 2404 enrich B cells from spleen and lymph nodes, 2405 stain B cells with HLA-neoepitope complexes, 2406 single-cell sorting of neoepitope specific B cells, 2407 RT-PCR and sequencing, 2408 clone (human) variable region sequences into human antibody vectors, 2409 transfect CHO cells and produce test batches of candidate antibodies, 2410 test Ab:Ag binding by ELISA, 2411 autologous NK cells, 2412 allogeneic NK cells (e.g. donor-derived cord blood-derived, iPSC-derived), 2413 irradiated NK cell line.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Antigen-binding fragment as used herein shall mean the part of an antibody that facilitates binding to its cognate antigen, including but not limited to the F(ab′)₂ fragment, Fab fragment, single-chain variable fragment (scFv), and single-domain antibody.

Immunoglobulin-humanized as used herein shall mean an animal that has undergone genetic modification such that its endogenous immunoglobulin genes are no longer expressed, and human immunoglobulin genes are expressed instead. These animals produce “fully human” antibodies, rather than “humanized” antibodies.

Neoantigen as used herein shall mean a protein which, as a result of somatic mutation, contains one or more changes in its amino acid sequence compared to the normal protein produced by cells lacking the mutation.

Neoepitope as used herein shall mean a peptide presented on cell-surface MHC molecules, derived from a neoantigen, and containing one or more of its changed amino acid residues.

Personalized as used herein shall mean developed individually for each patient.

While neoantigen-HLA (Human Leukocyte Antigen) complexes have been referenced in this invention for treating human patients, the relevance of neoantigen-MHC (Major Histocompatibility Complex) is also implicit (for example to treat non-human subjects).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention described herein is a novel approach to personalized cancer immunotherapy that uses monoclonal antibodies against tumor-specific neoepitope-HLA complexes (specific to each patient's cancer) to induce Fc gamma (Fcγ) receptor-expressing cytotoxic effector cells (endogenous and/or exogenously introduced by cell infusion) to kill cancerous cells in a highly specific manner. The invention includes the targeting of patients' tumor-specific neoantigens by leveraging antibody-dependent cellular cytotoxicity (ADCC), or antibody-dependent cellular phagocytosis (ADCP) by Fcγ receptor-expressing cytotoxic effector cells, rather than the more conventional dependence on the natural anti-neoantigen T cell response from the patients' own immune systems. The present invention overcomes the limitations and challenges of existing immunotherapy approaches by uniquely combining and leveraging several distinct concepts, including but not limited to: (1) targeting neoepitope-HLA complexes, (2) monoclonal antibodies against specific antigens combined with cytotoxic effector immune cells, and (3) the specific mechanisms of ADCC (or ADCP, or a combination of ADCC and ADCP) offered by endogenous immune cells or adoptive cellular therapy. Antibodies may be developed on a patient-by-patient basis, or on the basis of neoepitopes shared amongst a group of patients, against the mutated peptides that are presented by HLA molecules on cancer cells (these HLA-peptide complexes are normally targeted by the T cell receptor (TCR); therefore, the antibodies are “TCR-like”) (Dahan, R. & Reiter, Y. (2012) Expert Rev. Mol. Med. 14, e6). Infused Fcγ receptor-expressing cytotoxic effector cells (e.g. NK cells expressing CD16, macrophages expressing CD32) are directed by these TCR-like antibodies to specifically attack each particular patient's cancer cells, primarily leveraging the mechanisms of ADCC, or ADCP (or both ADCC and ADCP). The present invention is described in enabling detail in the following examples, which may represent more than one embodiment of the present invention.

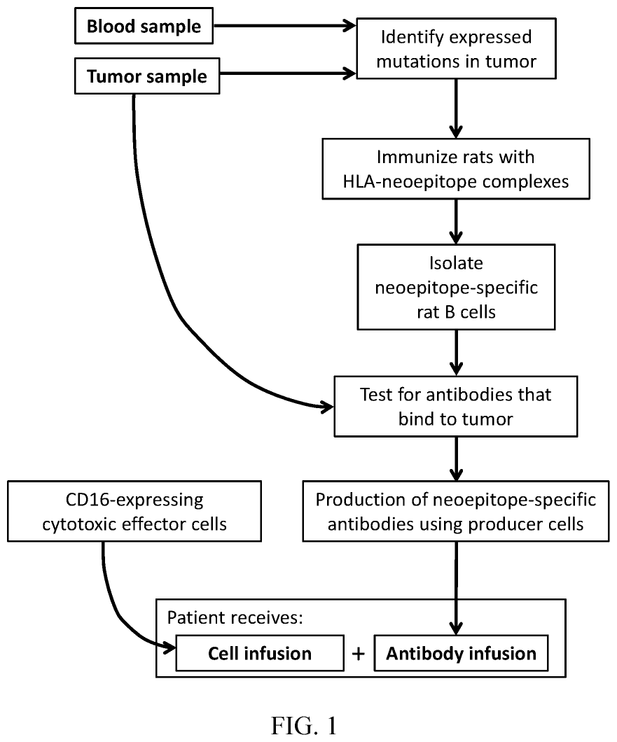

FIG. 1 is a pictorial representation of an embodiment of the invention. Beginning with freshly isolated tumor tissue following surgery or biopsy, mutations may be identified by comparing the DNA sequences of tumor tissue and normal tissue (blood). Next-generation sequencing platforms such as Illumina and/or Ion Torrent may be used individually or combined—by sequencing with two different platforms and only using mutations detected by both, confidence in the identified mutations is high (Sherafat, et al. (2020) BMC Bioinformatics 21: 498). Mutated peptides, complexed with HLA molecules, may be used to immunize rats. Rat B cells producing TCR-like antibodies specific for the neoepitope-HLA complexes may be isolated, and the antibodies may be tested for binding to the tumor cells (Ouisse, (2017) BMC Biotechnol. 17, 3). Those antibodies which test positive for tumor-specific binding may be produced in larger quantities by producer cells (e.g. CHO, HEK-293). The resulting monoclonal antibodies targeting tumor-specific mutations may be administered to the patient, either alone or along with an infusion of Fcγ receptor-expressing cytotoxic effector cells.

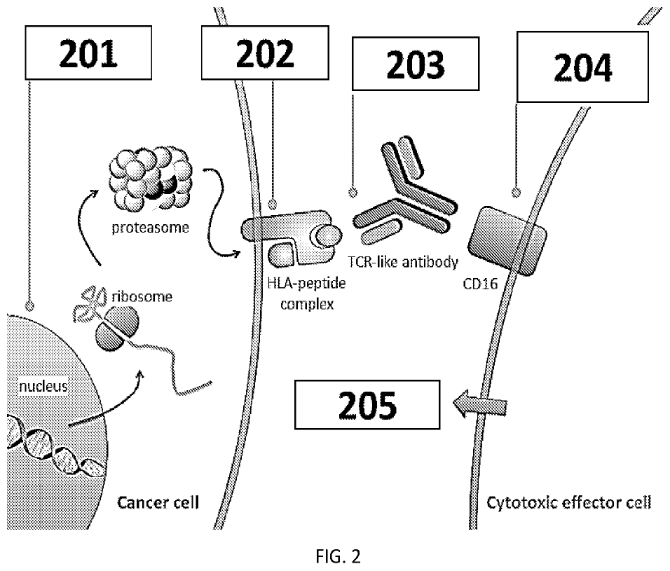

Because TCR-like antibodies targeting tumor-specific neoepitopes may be administered along with Fc-gamma receptor-expressing cytotoxic effector cells, the cytotoxic action of the infused cells is directed specifically against tumor cells. The mechanism of this embodiment of the invention is depicted in FIG. 2.

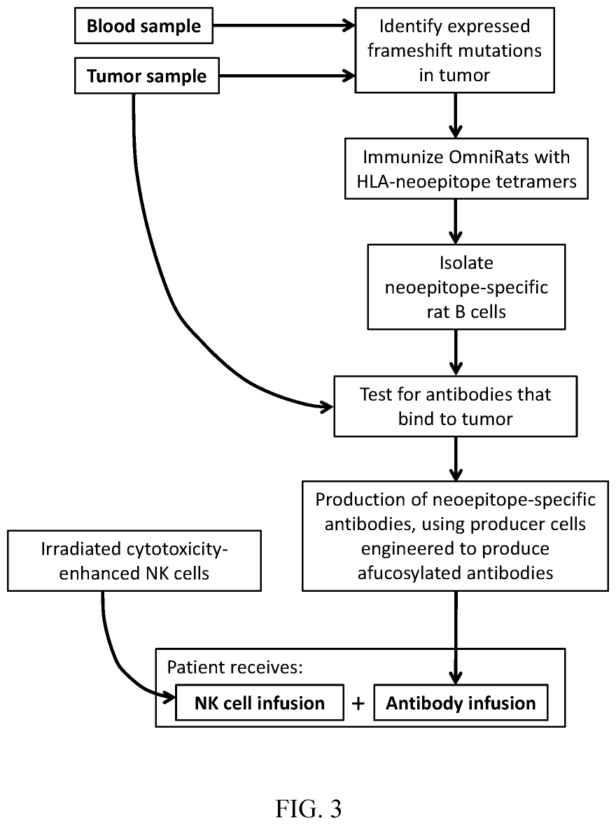

In one embodiment, which is the preferred embodiment, the particular category of neoepitope targeted by the TCR-like antibodies is that derived from insertion/deletion (InDel) mutations resulting in frameshifts. The neoepitopes that arise from frameshift mutations are entirely foreign; unlike neoepitopes arising from single nucleotide variant (SNV) mutations, there is no associated “self” counterpart epitope. Thus, by targeting frameshift-derived neoepitopes, the risk of antibody cross-reactivity with normally presented “self” epitopes is averted. In the preferred embodiment, immunoglobulin-humanized rats (“OmniRats”) are used for immunizations to generate neoepitope-specific B cells. This leads to the production of “fully human” antibodies, minimizing the risk of adverse events in patients. In the preferred embodiment, the final antibodies to be delivered to the patient are produced by producer cells engineered to generate afucosylated antibodies, which lead to enhanced cytotoxic activity by effector cells. In the preferred embodiment, the effector cells are natural killer (NK) cells, cultured in media to enhance their cytotoxic activity, and irradiated prior to administration to the patient. In FIG. 3, these aspects of the preferred embodiment are incorporated into the same flow chart seen in FIG. 1.

This preferred embodiment of the invention is intended to combine the most promising aspects of disparate research avenues and therapeutic approaches uniquely and optimally—mutated peptide neoepitopes, targeted therapy using monoclonal antibodies, and adoptive NK cell therapy—into a novel approach with personalized antibody therapy that acts in concert with cellular therapy, and is more effective and less toxic overall.

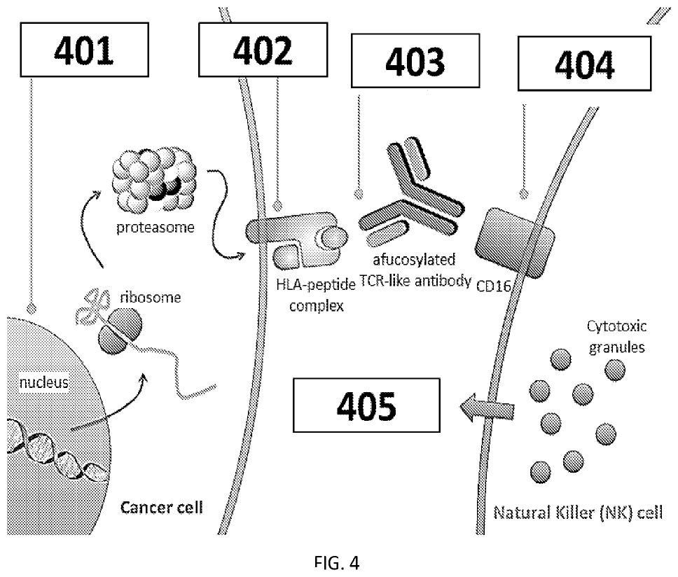

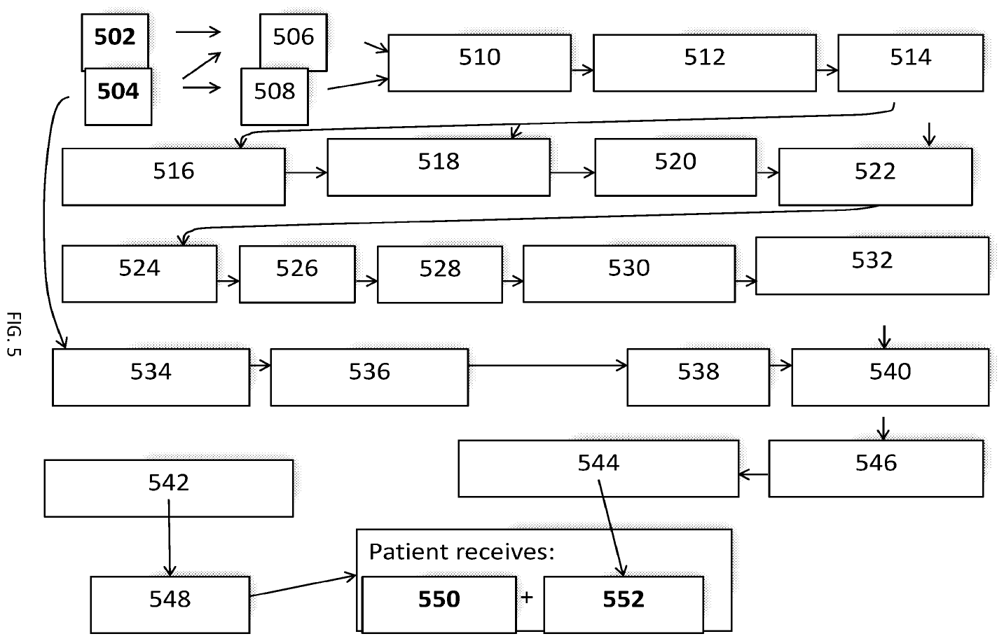

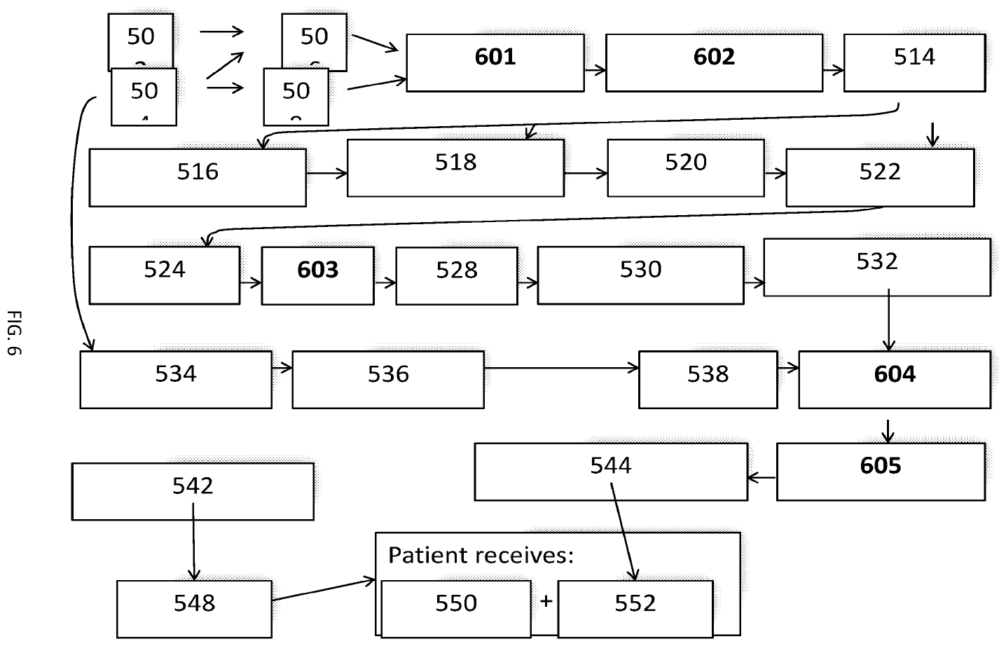

The mechanism of action of one embodiment, which is the preferred embodiment depicted in FIG. 3, is depicted in FIG. 4. Certain cancer types (e.g. renal cell carcinoma) tend to have, on average, a high number of InDel mutations—these high-InDel cancers therefore have the highest likelihood of having frameshifts in protein-coding genes (Bailey, et al. (2018) Cell 173: 371-385.e18)(Turajlic, et al. (2017) Lancet Oncol. 18: 1009-1021). Frameshifts often result in extended sequences of amino acids that are completely novel—that is, they are unlike any amino acid sequence seen in normal human cells. When these novel sequences undergo antigen processing (e.g. proteasomal degradation) and presentation on cell-surface HLA molecules, they constitute truly cancer-specific epitopes (“neoepitopes”) that are completely unlike any HLA-presented “self” epitope, and therefore represent a unique target for personalization of therapy (Linnebacher, et al. (2001) Int. J. Cancer. 93: 6-11.) In the preferred embodiment, high-InDel cancer types are the main candidates for the immunotherapy, and frameshift-derived neoepitopes are the preferred targets for the monoclonal TCR-like antibody development. This is due to the fact that frameshift-derived neoepitopes, unlike SNV-derived neoepitopes, lack a similar “self” peptide counterpart sequence that may be presented by normal cells; frameshift-derived neoepitopes therefore offer the prospect of higher specificity of directed therapy against cancer cells while leaving normal cells unaffected. The variable region of the antibody binds the neoepitope-HLA complex, and the constant region (i.e. the Fc region) is available to be bound by Fc receptors such as CD16, or CD32, or CD64. In the preferred embodiment, the Fc region is afucosylated, having been produced by producer cells engineered to generate afucosylated antibodies. (This modification greatly increases cytotoxic activity by effector cells.) The CD16 molecule on the surface of the infused NK cells triggers the cytotoxic activity, consisting of cytokine secretion and/or the release of cytotoxic granules, killing the tumor cell. The cytotoxic activity, in the preferred embodiment, is antibody-dependent cellular cytotoxicity (ADCC).

The sequence of steps in the process of producing the personalized treatment protocol, is outlined below. It is depicted as a detailed flow chart in FIG. 5. FIG. 5 legends correspond as follows: 502 Blood Sample, 504 Tumor sample, 506 Next-generation DNA sequencing, 508 RNA sequencing, 510 Identify expressed frameshift mutations in tumor, 512 Predict frameshift-derived high-affinity neoepitopes based on HLA haplotype, 514 Produce HLA neoepitope tetramers, 516 Immunize OmniRats with HLA-neoepitope tetramers, 518 Immunize OmniRats with HLA-neoepitope tetramers a second time, 520 Enrich B cells from spleen and lymph nodes, 522 Stain B cells with HLA-neoepitope tetramers, 524 Single cell sorting of neoepitope-specific B cells, 526 Test Ab:Ag by ELISA524, 528 RT-PCR and sequencing of positives, 530 Clone (human) variable region sequences into human antibody vectors, 532 Produce small test-batches of candidate anti-neoepitope antibodies using producer cell lines, 534 Create single-cell suspension of patient's tumor cells, 536 Cryopreservation, 538 Thaw patient's tumor cells, 540 Stain tumor cells with candidate anti-neoepitope antibodies by ELISA, 542 Culture an NK cell line (that expresses CD16) in cytoxicity-enhancing media, 544 Larger-scale GMP production of successful antibodies, using producer cells engineered to produce afucosylated antibodies, 546 Successful tumor-binding HLA-neoepitope-specific antibodies are selected, 548 Irradiate NK cells, 550 NK cell infusion, and 552 Antibody infusion.
(1) Surgical resection or biopsy of primary tumor, dissociation into single-cell suspension, and cryopreservation (for use in step 8).
(2) Sequencing of blood and tumor tissue to identify expressed tumor-specific InDel mutations that result in frameshifts. Next-generation sequencing platforms such as Illumina and/or Ion Torrent can be used individually or combined.
(3) Algorithmic prediction of neoepitopes with high affinity for the patient's HLA class I molecules. The open-source NetMHC prediction algorithms, for example, accurately predict HLA class I epitopes for all 12 HLA-A and -B supertypes using artificial neural networks.
(4) Production of HLA-neoepitope tetramers (for steps 5 and 6). Several companies (e.g. GenScript, JPT) synthesize custom peptides. Several companies (e.g. MBL International, ProImmune) produce custom HLA-peptide tetramers, and/or provide kits.
(5) Immunization of immunoglobulin-humanized rats (“OmniRats”) with HLA-neoepitope tetramers to generate B cells with TCR-like neoepitope specificity. The immunization protocol of 100 μg HLA-peptide complex in Complete Freund's Adjuvant, followed by 100 μg HLA-peptide complex in PBS 16 days later, has been shown to generate high-affinity anti-HLA-peptide antibodies in rats, 5 days after the second immunization7.
(6) Isolation (using tetramer staining and cell sorting) of HLA-neoepitope-specific rat B cells, and sequencing of the antibody genes (heavy and light chains).
(7) Initial testing of the binding of candidate antibodies to the HLA-neoepitope complexes using ELISA (kits for custom ELISAs are commercially available).
(8) Cloning of successful antibody variable sequences into human antibody plasmid backbones (commercially available from several companies, e.g. InvivoGen), transfection of producer cells (e.g. CHO, HEK-293), and small-scale production of several candidate anti-neoepitope antibodies to be tested.
(9) Thawing of cryopreserved tumor cells, and staining with candidate antibodies to test for tumor-specific binding.
(10) Larger-scale production (GMP) of tumor cell-binding antibodies, using producer cells engineered to produce afucosylated (ADCC-optimized) antibodies.
(11) Treatment administered to patient: Infusion of NK cells (grown under conditions to maximize cytotoxic activity) along with infusion of anti-neoepitope TCR-like antibodies.

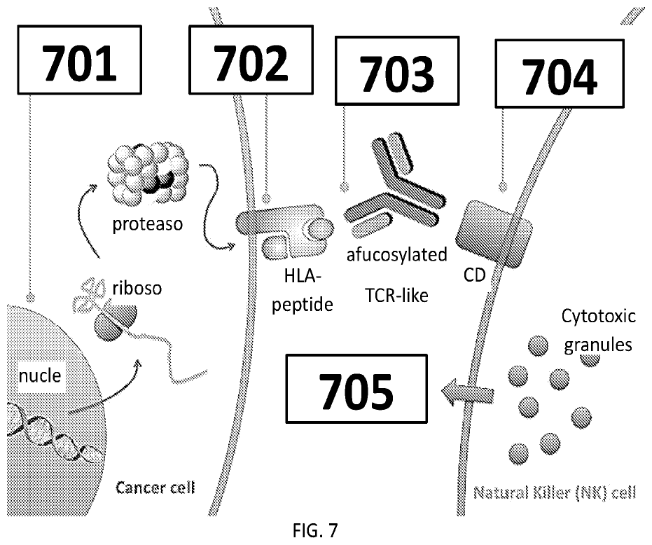

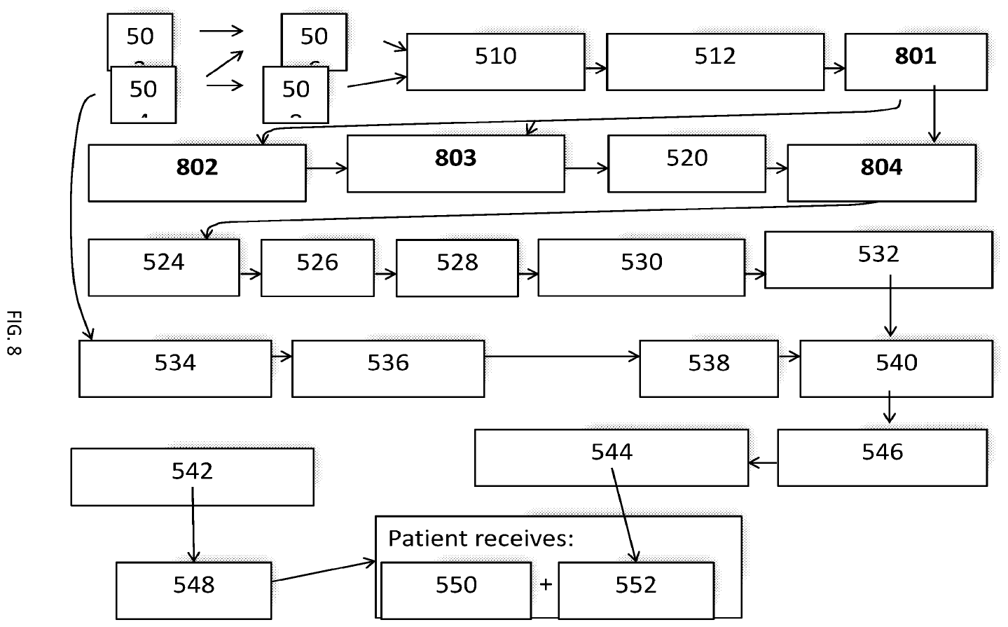

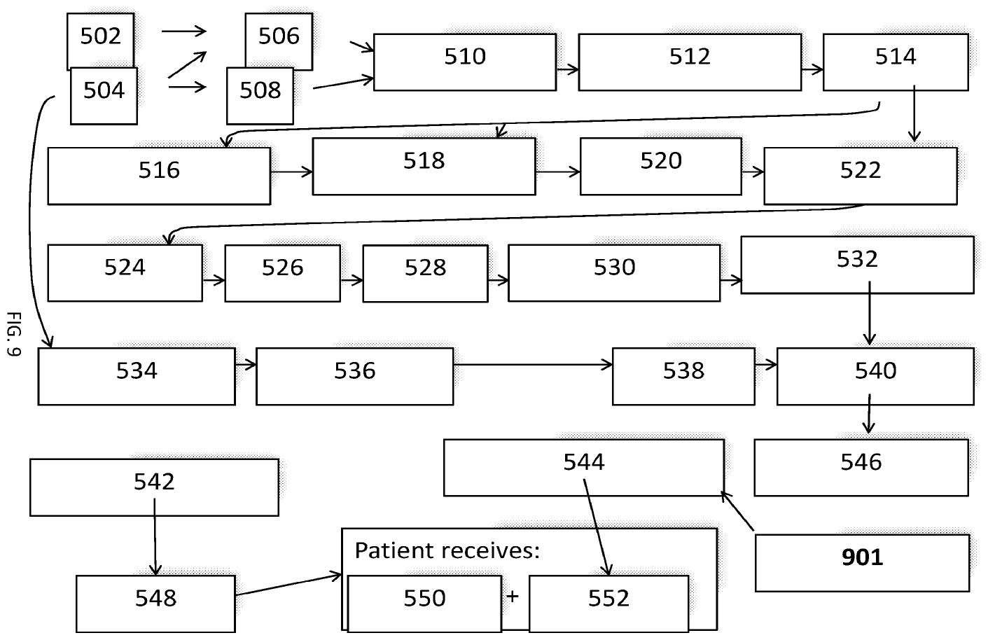

There are several potential modifications to the preferred embodiment, which constitute other embodiments of the present invention. In one embodiment, SNV-derived neoepitopes are identified in the tumor. While these neoepitopes have “self” counterpart epitopes that differ by only one amino acid residue, and therefore raise the possibility of crossreactivity of the TCR-like antibodies against normal tissue, this challenge is overcome by adding further rounds of specificity testing to the process (see FIGS. 6 and 7). Additionally, neoepitopes arising from any other type of mutation (e.g. translocations), or from dysfunctional RNA splicing, or from any other mechanism that is tumor-specific, constitute candidate targets for this therapy.

In another embodiment, neoepitopes presented by HLA classes other than class I (e.g. HLA class II) constitute the targets for the therapy.

In another embodiment, instead of HLA-presented neoepitope peptides, the tumor-specific molecules targeted by the therapy are mutated cell surface molecules (e.g. receptors, adhesion molecules), where the mutations have created substantive tumor-specific changes in the structure of the extracellular portions of the molecules, to an extent that they can be distinguished from their unmutated counterparts by antibodies.

Embodiments of this invention include mutations of any kind/source, for example somatic mutations, passenger mutations and others.

In another embodiment, the tumor-specific molecular targets of the therapy are not changes in amino acid sequences per se, but instead are changes in post-translational modifications, including but not limited to glycosylation patterns and acetylation. Included are neoantigens that arise due to a combination of amino acid sequence changes along with post-translational modifications.

In another embodiment, the HLA-epitope complexes used to immunize the rats, and/or those used to isolate specific rat B cells, and/or those used to test antibody binding, are either monomeric, pentameric, or dextran-linked, instead of tetrameric (see FIG. 8).

In another embodiment, the immunized rats are a strain of rat other than OmniRats.

In another embodiment, the immunized animal is an animal other than a rat, including but not limited to a mouse, a rabbit, a donkey, a goat, a camel, a llama, an alpaca, and a shark.

In another embodiment, the tumor-specific antibodies are generated by a means other than immunizing animals and sorting their antigen-specific B cells, including but not limited to the use of phage display libraries, and antibody gene transfer by injection of antibody-encoding nucleic acid (e.g. DNA) into the patients.

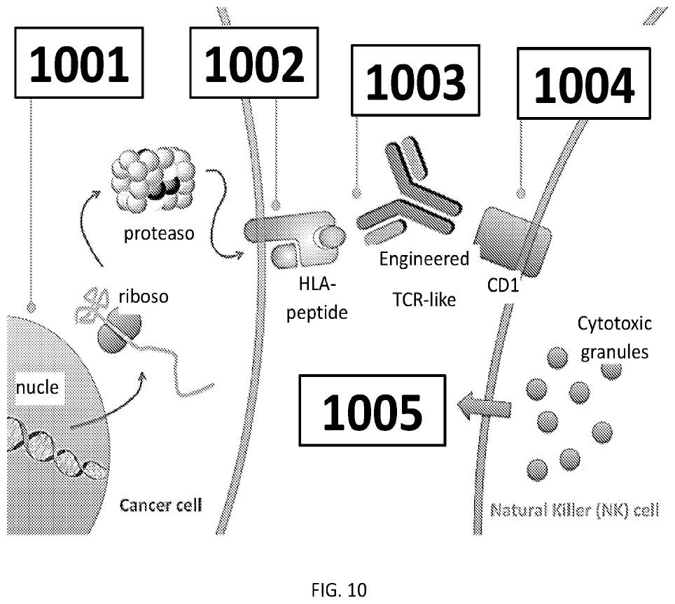

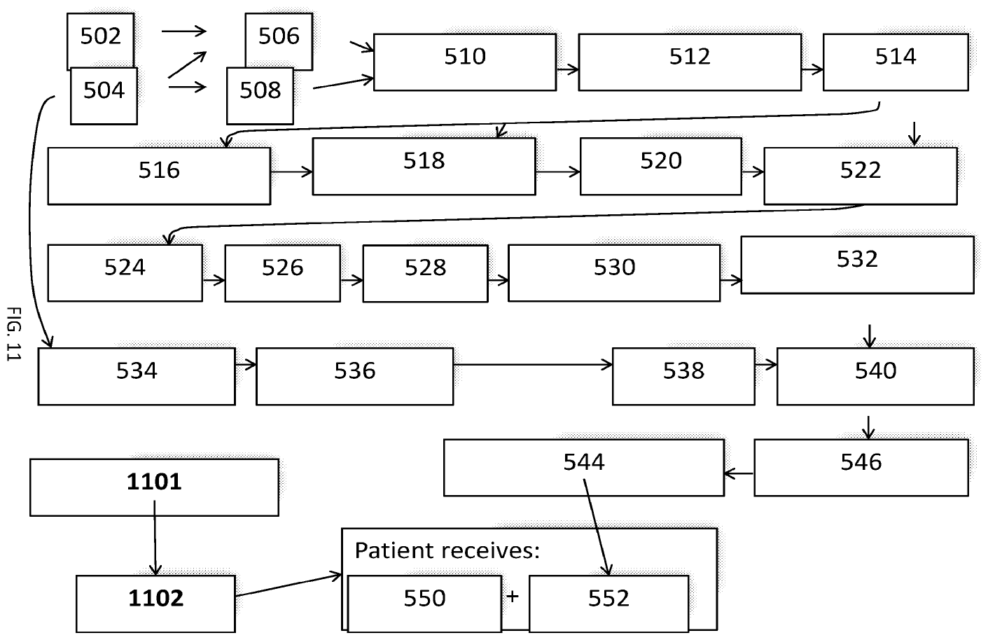

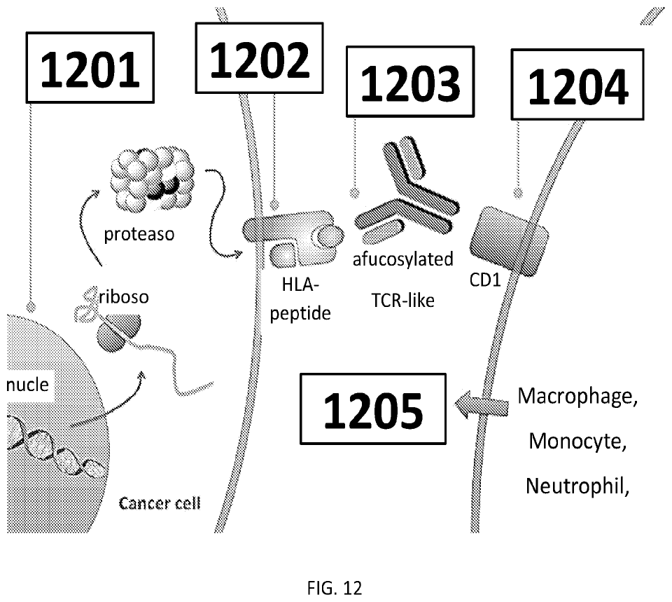

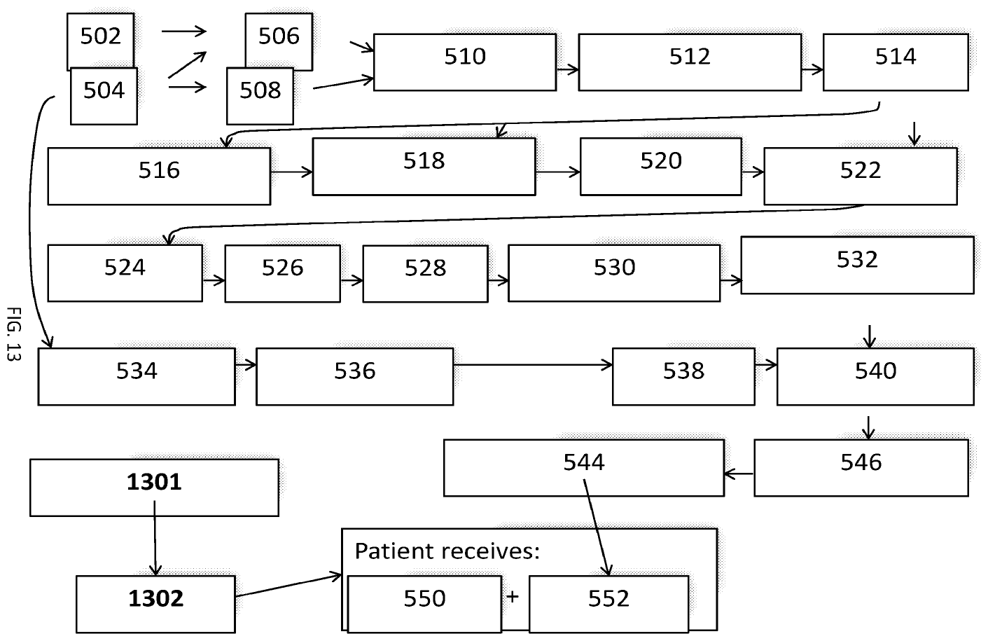

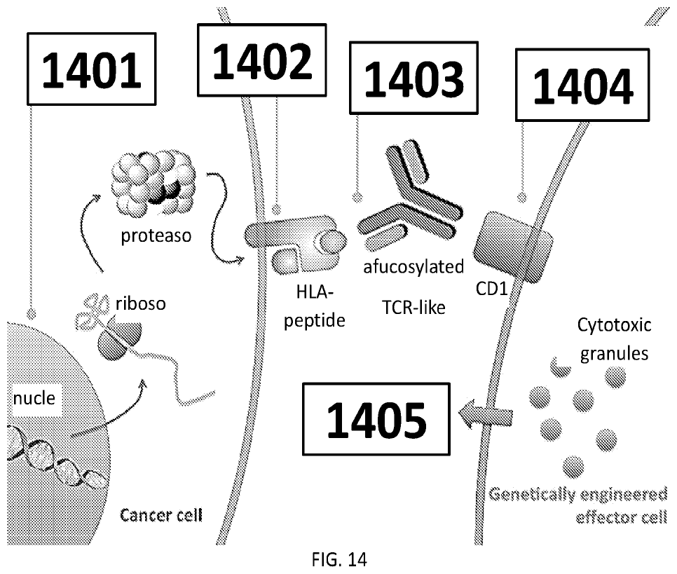

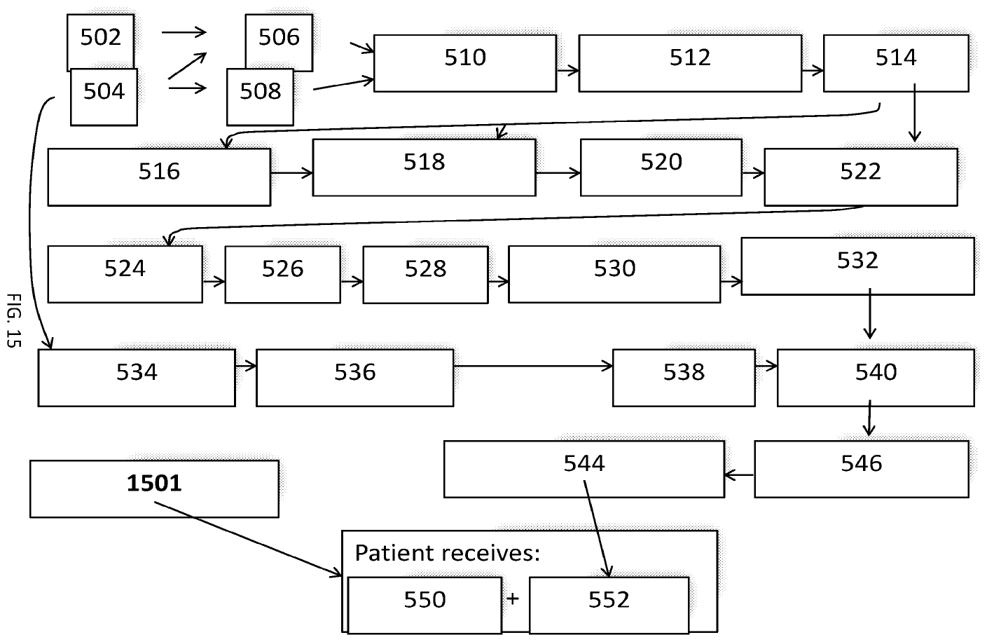

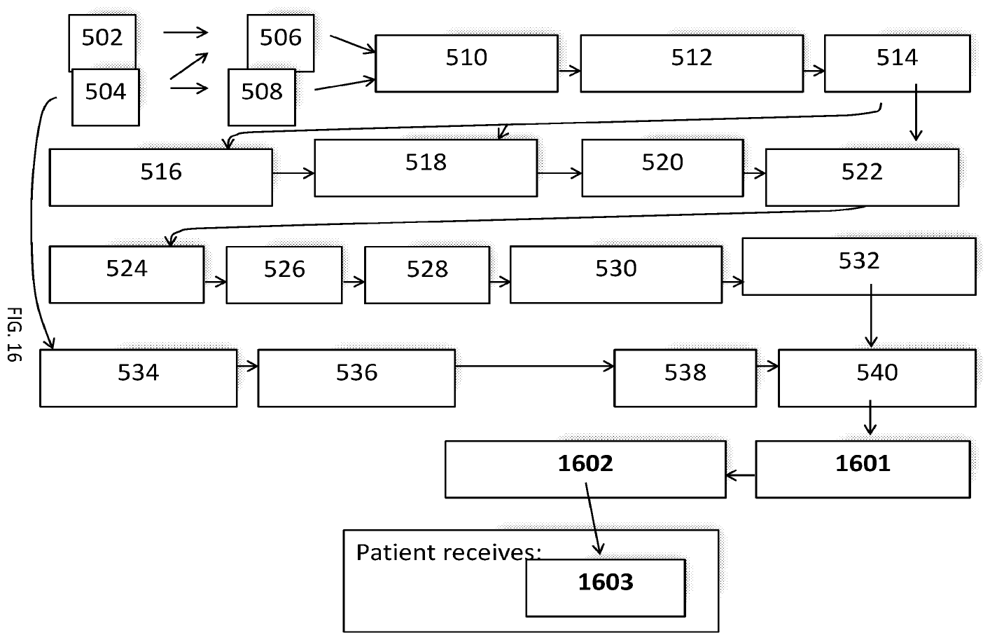

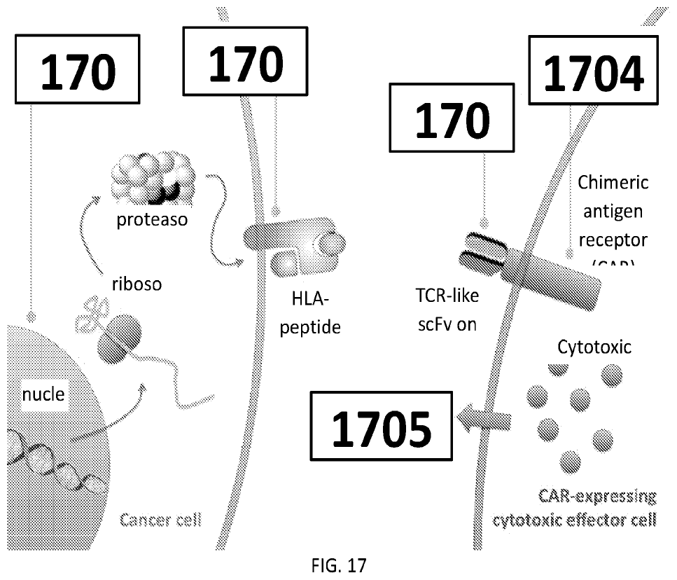

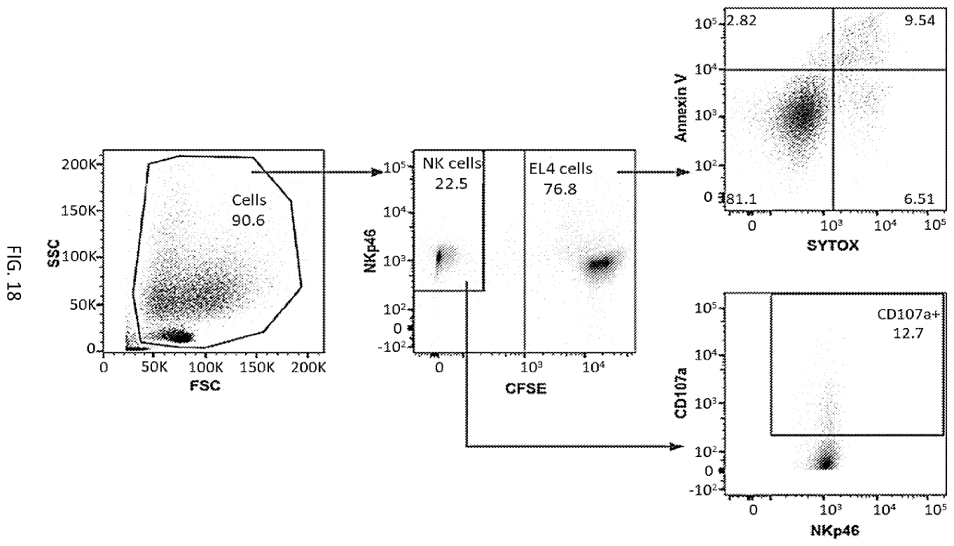

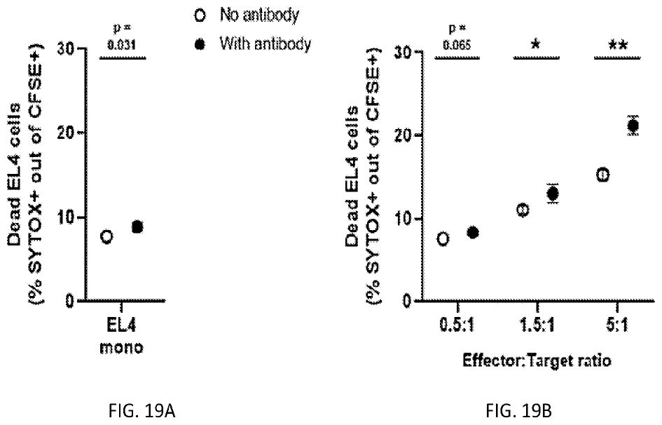

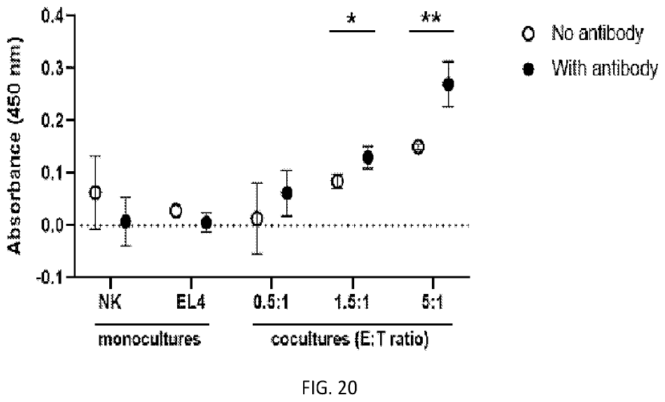

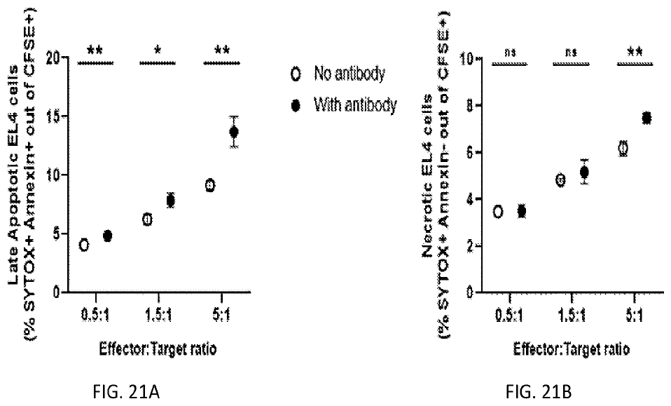

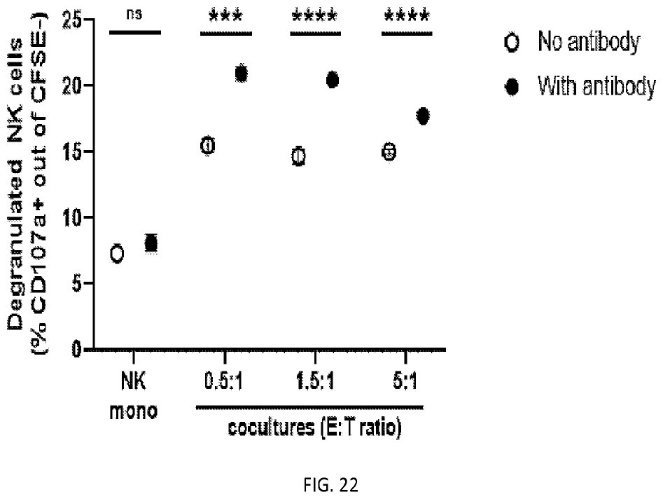

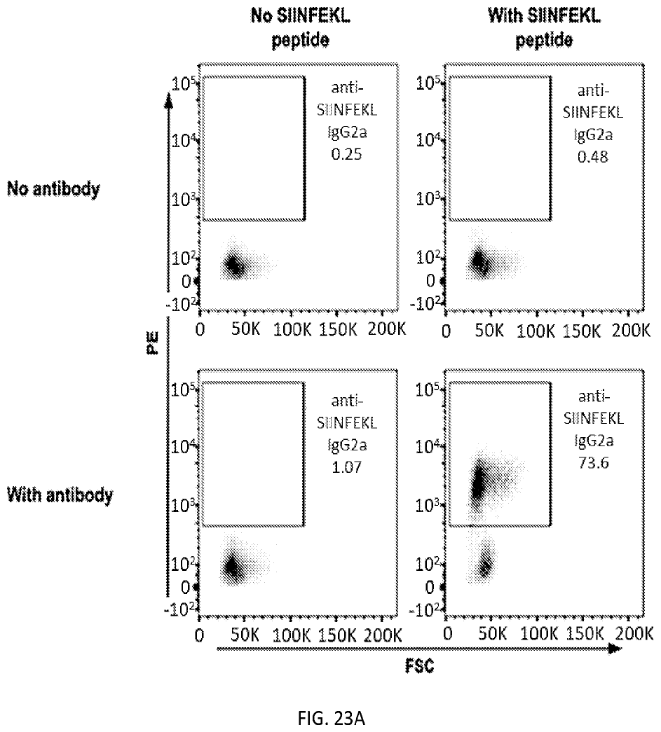

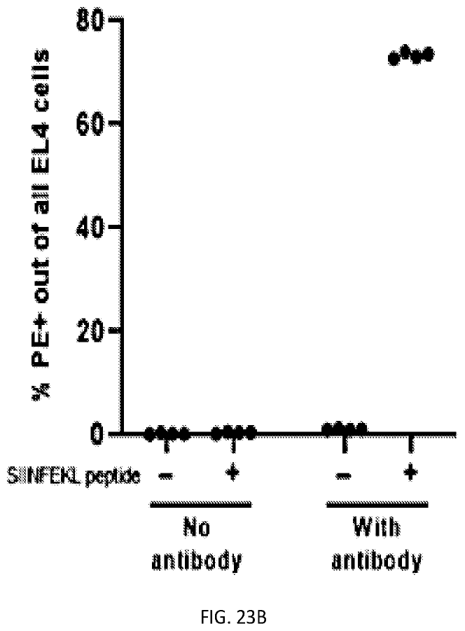

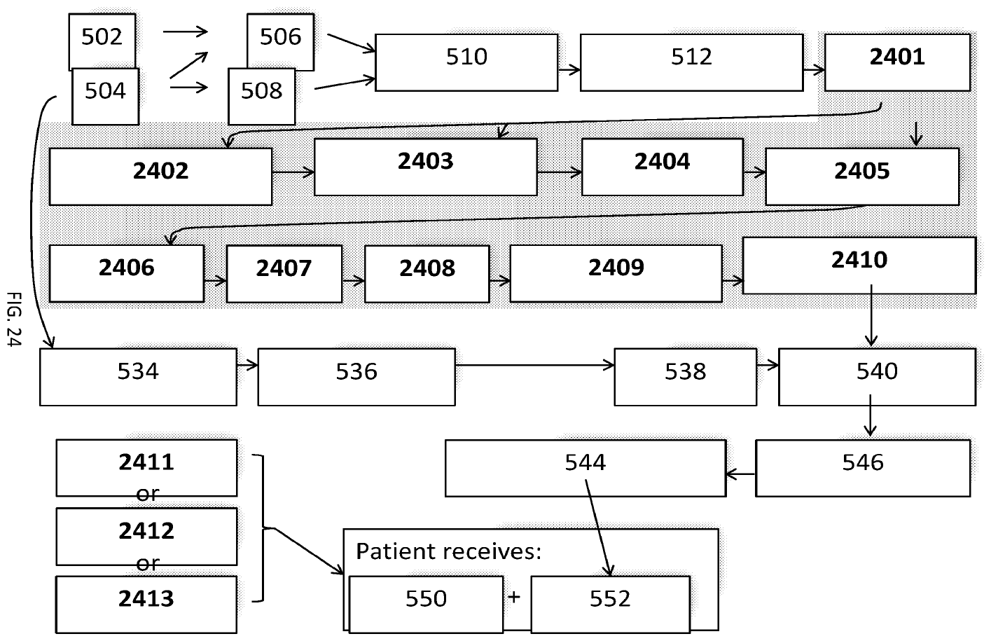

In another embodiment, the TCR-like antibodies are engineered or produced differently than in the preferred embodiment. Rather than simply employing afucosylation as a means to increase ADCC activity, the Fc region of the antibodies could be altered in other ways (e.g. the engineering of disulfide bonds within the antibody structure; Hagihara, Y. and Saerens, D., (2014). Biochimica et Biophysica Acta (BBA)—Proteins and Proteomics, 1844(11), pp. 2016-2023.). At the same time, the variable region may also be engineered in various ways, for example, the antibody may be bispecific (see FIGS. 9 and 10), where the bispecific antibody may be directed against two distinct neoepitopes, or the combination of a neoepitope and a regular epitope (i.e. an epitope that is not a neoepitope). Alternatively or in addition, the antibody could be conjugated to a drug, such as an anti-cancer drug, i.e. antibody-drug conjugates. Other embodiments include methods (e.g. molecular methods) to modify/engineer antibodies.

In another embodiment, the neoepitope-specific antibodies used in the therapy are polyclonal instead of monoclonal.

In another embodiment, the neoepitope-specific antibodies are not full antibodies, but instead are antigen-binding fragments, including but not limited to the F(ab′)2 fragment, Fab fragment, single-chain variable fragment (scFv), and single-domain antibody.

In certain embodiments of the invention, cell types other than NK cells may be leveraged (endogenous or exogenously administered) in concert with the neoepitope targeting antibody. The therapy depends on the interaction between the Fc region of the TCR-like antibody and the Fcγ receptor on the surface of the effector cells. Certain cell types, including but not limited to cells of the innate immune system such as macrophages, monocytes, neutrophils, and dendritic cells which express one of, or a combination of, the Fcγ receptors CD16, CD32, and CD64; these cell types could alternatively serve as the effector cells in the therapy (see FIGS. 11 and 12). Implicit in these alternative embodiments is the option of using subsets or subpopulations of these cell types, for example memory NK cells.

In certain embodiments, cell types other than cells of the innate immune system may be used (in concert with the neoepitope targeting antibody), for example the following cell types which are also known to express Fcγ receptors: activated T cells, endothelial cells, microglial cells, osteoclasts, and mesangial cells.

In certain embodiments of the invention, the target disease cell may manifest a neoepitope in the context of disease(s) other than cancer, including but not limited to autoimmune diseases.

It is implicit in this detailed description that the neoepitope-specific antibodies used in the therapy will be of isotype IgG, because Fcγ receptors bind to the constant region of IgG antibodies. The specific subclass of IgG (e.g. IgG1, IgG3, etc.) that is chosen for therapeutic use will depend on the mechanism of action intended (e.g. ADCC, ADCP) and the cell types being employed in the therapy. Several different isotypes or isotype subclasses may be used individually or combined in a particular therapy.

In another embodiment, the isotype (or subclass) of the antibody is switched from an inactive or undesired isotype (or subclass) to one with the desired properties.

In certain embodiments of the invention, the following scenarios are included: the same antibody and even the same effector cell (e.g. monocyte) can induce both ADCC and ADCP (Strohl, W. and Strohl, L., 2012. 8-Monoclonal antibody targets and mechanisms of action. Thera Antibody Eng, 9, pp. 163-196.). Also, different antibodies against the same target can induce various mechanisms, including ADCC and ADCP (Cleary, K. L., Chan, H. C., James, S., Glennie, M. J. and Cragg, M. S., 2017. Antibody distance from the cell membrane regulates antibody effector mechanisms. The Journal of Immunology, 198(10), pp. 3999-4011). Additionally, different antibody isotypes can evoke and elicit ADCC and ADCP mechanisms differently (Tipton, T. R., Roghanian, A., Oldham, R. J., Carter, M. J., Cox, K. L., Mockridge, C. I., French, R. R., Dahal, L. N., Duriez, P. J., Hargreaves, P. G. and Cragg, M. S., 2015. Antigenic modulation limits the effector cell mechanisms employed by type I anti-CD20 monoclonal antibodies. Blood, The Journal of the American Society of Hematology, 125(12), pp. 1901-1909.).

In certain embodiments of the invention, a set of antibodies (whether or not from an antibody library) may be generated against a given neoepitope.

In another embodiment, the infused effector cells are genetically engineered. These manipulations may include, but are not limited to, knocking out or knocking down certain genes, overexpression of certain genes, or expression of exogenous genes (see FIGS. 13 and 14).

In another embodiment, the infused effector cells are obtained from one of several potential sources. NK cells could be sourced from allogeneic donors, cord blood, or the patient's own blood; alternatively, several NK cell lines could potentially be used, provided that they express CD16, and are irradiated prior to administration to the patient (see FIG. 15).

In another embodiment, the production of whole antibodies is replaced with the production of a corresponding scFv sequence, followed by cloning into a chimeric antigen receptor (CAR) construct. Effector cells are then engineered to express the CAR construct prior to infusion. In this embodiment, administration of TCR-like antibodies against neoepitopes may be used in combination with CAR effector cells, or the TCR-like antibodies may not be part of the therapy protocol, with the antigen specificity provided by the membrane-bound CAR (see FIGS. 16 and 17). Implicit in FIGS. 16 and 17 is the option of including the administration of TCR-like antibodies generated against the neoepitope-HLA complexes.

Certain embodiments of the invention may leverage therapeutic mechanisms of antibodies which are independent of ADCC or ADCP, for example CDC (Complement Dependent Cytotoxicity).

Certain embodiments of the invention include treating the same patient with more than one antibody generated against newer neoepitopes being manifested in the patient's body over time, for example different neoepitopes appearing in different tumors during the process of cancer metastasis.

Each of the embodiments described in this Detailed Description section are also applicable to mutations, and the resulting neoepitopes, that are shared between two or more patients (also called “public neoantigens” and “public neoepitopes”).

Exemplary methods of Use: The invention can potentially be used for treatment of any cancer for which a biopsy or surgical sample can be obtained. The most promising treatment candidates are those cancers which contain, on average, a high number of InDel mutations. The frameshift-derived neoepitopes that result from InDel mutations are most likely to enable the production of successful tumor-specific antibodies that lack cross reactivity with “self” epitopes. Thus, one series of embodiments focuses on high-InDel cancers and their associated frameshift-derived neoepitopes. However, certain embodiments target other types of mutations, e.g. single nucleotide variants (SNVs), as well.

One series of embodiments focuses upon cancers which tend to contain high numbers of InDels, including but not limited to kidney cancers, melanoma, bladder urothelial carcinoma, lung adenocarcinoma, uterine carcinosarcoma, uterine corpus endometrial carcinoma, breast cancer, head and neck cancers, and stomach adenocarcinoma.

For the embodiment that instead targets SNVs, any cancer with expressed SNV mutations in the exome would be a candidate for this immunotherapy. This would include the vast majority of cancers.

Overview: The invention includes the targeting of patients' tumor-specific neoantigens by leveraging antibody-dependent cellular cytotoxicity (ADCC), or antibody-dependent cellular phagocytosis (ADCP) by Fcγ receptor-expressing cytotoxic effector cells, rather than the more conventional dependence on the natural anti-neoantigen T cell response from the patients' own immune systems. There are thus key distinctions to the present invention as compared to existing immunotherapy approaches:
Unlike other targeted therapies using monoclonal antibodies, the present invention produces antibodies that bind only to tumor cells: Existing targeted monoclonal antibody treatments target cell-surface molecules such as CD20 (e.g. rituximab), EGFR (e.g. cetuximab), HER2 (e.g. trastuzumab), and several others. Although in many cases the targeted antigen is overexpressed on cancer cells, it is never expressed exclusively on cancer cells. Neoepitopes arising from mutations in cancer cells are presented exclusively on cancer cells, and it follows that TCR-like antibodies specific for those neoepitopes would only bind with high affinity to cancer cells.
Unlike other NK cell-based (or macrophage-based, or neutrophil-based) immunotherapies, the present invention targets the infused cells to the cancer cells in a highly tumor-specific manner: Numerous cell-based therapies are undergoing clinical trials, but none of these approaches are based on targeting the effector cells to cancer cells in a highly specific manner. Since the present invention targets effector cells to cancer cells using antibodies as bridges that only bind to cancer cells, a much higher level of effector specificity is achieved.
Unlike other attempts to utilize the high tumor-specificity of neoepitopes, the present invention does not rely on the patient's natural T-cell immune response: Research efforts by other groups have aimed at developing therapeutic immunizations, on a patient-by-patient basis, intended to initiate (or “reinvigorate”) a T cell response against neoepitopes presented by cancerous cells. These efforts have run into several challenges, and significant clinical success has not been achieved. The present invention eschews these challenges by targeting cancer cells in a way that does not rely on the patient's T cell response. Instead, it uses externally-generated antibodies to target the neoepitopes, combined with externally-generated (i.e. externally cultured, genetically modified, expanded, and activated) effector cells. In contrast to immunization based therapeutic efforts using neoepitopes and neoantigens, the current invention therefore makes the outcome of therapy more predictable and targeted, given its reliance on both antibody recognition of neoepitopes, as well as innate immune effector cell functions of ADCC or ADCP (or combination of ADCC and ADCP).
Unlike work by some other groups, our invention is not centered on any specific neoepitope, and can therefore be applied (e.g. in a personalized manner) to any neoepitope either already known, or to neoepitopes which will be discovered in the future.
Unlike work by some groups generating transgenic TCRs or CARs that target neoepitopes, the present invention can be more easily developed on a patient-by-patient basis to produce truly personalized therapies: Other research and development groups, also looking to harness the exquisite cancer specificity of neoepitopes, have developed transgenic neoepitope-specific TCRs, and/or neoepitope-specific CARs. In each of these cases, the cytotoxic effector cells (whether NK cells or otherwise) would need to be transduced with the neoepitope-specific construct when there is no dependence on antibody-based recognition of the neoepitope. In contrast, the present invention relies on antibody-based recognition of the neoepitopes, while also specifically leveraging the ADCC or ADCP mechanisms of those immune effector cells, which can support ADCC or ADCP through Fc-gamma receptors (e.g. CD16 and CD32 receptors respectively for ADCC and ADCP). Because the personalization relies on the generation of personalized antibodies rather than personalized genetically modified immune effector cells, the present invention also offers better efficiency in the generation of personalized therapy. The current invention offers the unique combination of [a] more time-efficient and resource-efficient antibody based personalization of therapy, along with [b] the effectiveness of immune effector cells working in concert with antibodies to more selectively target cancer cells over normal cells—by virtue of the targeting of neoepitope-HLA complexes by antibodies which bridge adoptively transferred immune effector cells to target cancerous cells.

It will be apparent to one with skill in the art that the personalized cancer immunotherapies of the invention may be provided using some or all of the mentioned features and components without departing from the spirit and scope of the present invention. It will also be apparent to the skilled artisan that the embodiments described above are specific examples of a single broader invention which may have greater scope than any of the singular descriptions taught. There may be many alterations made in the descriptions without departing from the spirit and scope of the present invention.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Antibody Against Neoepitope Induces Natural Killer (NK) Cell Mechanism of ADCC [Antibody Dependent Cell-Mediated Cytotoxicity] to Kill Target Cells Presenting the Neoepitope

While neoepitopes constitute attractive targets for cancer immunotherapy (Brennick et al. (2017) Immunotherapy, 9(4), pp. 361-371), the novel methodology disclosed herein to generate antibody therapeutics directed against neoepitopes is centered on two specific cellular mechanisms manifested primarily by cells of the innate immune system which leverage antibodies as a bridge to neoepitopes on the surface of cancer calls, namely the mechanisms of ADCC and ADCP [Antibody Dependent Cell-mediated Cytotoxicity and Antibody Dependent Cell-mediated Phagocytosis](Gogesch et al. (2021) International Journal of Molecular Sciences, 22(16), p. 8947). As proof-of-concept or proof-of-principle, this experimental study illustrates that TCR-like antibodies generated against neoepitopes can induce ADCC cytotoxic activity of Natural Killer (NK) effector cells towards target cells presenting those specific neoepitopes. In the experimental data summarized below, the model peptide epitope SIINFEKL served as a “model neoepitope” or “surrogate neoepitope.” That is, for the purpose of proof-of-concept experiments, antibody-mediated NK cell responses against the surrogate neoepitope SIINFEKL are considered mechanistically equivalent to the envisioned antibody-mediated NK cell responses against neoepitopes in diseased cells (such as cancer cells), especially those neoepitopes which are derived from frameshift mutations and result in completely novel or foreign peptide sequences.

Experimental methodology: The anti-SIINFEKL-H2K^{b} antibody (clone 25.D1-16) is a commercially available “TCR-like antibody” (that is, it binds to a peptide-MHC complex much like a TCR does). This antibody was chosen for preliminary proof of concept experiments due to its well-established use in detecting the SIINFEKL-H2K^{b} complex (with SIINFEKL representing a model neoantigen or neoepitope). However, because this mouse antibody's isotype is IgG1 (which does not induce ADCC activity), it was necessary to switch its isotype to the ADCC-active mouse IgG2a isotype for these experiments. The switch to IgG2a was performed by cloning the variable region sequences of both the heavy chain and light chain of the original 25.D1-16 antibody into a mouse IgG2a plasmid (pTRIOZ-mIgG2a) commercially available from Invivogen (San Diego, CA). Production of the plasmid, and subsequent recombinant expression of the antibody protein in the Chinese Hamster Ovary (CHO) cells, were performed by Azenta (Chelmsford, MA). Protein purification was performed using Protein A agarose.

To test the induction of ADCC by mouse NK cells via the anti-SIINFEKL-H2K^{b} TCR-like antibody, cocultures were set up at 3 different Effector:Target (E:T) ratios for a period of 4 hours using primary mouse NK effector cells and EL4 target cells (a non-adherent C57BL/6 mouse cell line expressing H2K^{b}). Before the coculture setup, EL4 cells with >95% viability were labeled with the CarboxyFluorescein Succinimidyl Ester (CFSE) stain, (to provide a clear means of distinguishing targets from effectors) at a staining concentration of 0.25 μM, and incubated (i.e. “pulsed”) with 25 μM SIINFEKL peptide for 1 hour at 37° C., with mild agitation every 15 minutes. These CFSE-stained, SIINFEKL-pulsed EL4 cells were then plated in a round-bottom 96-well plate at 50,000 cells per well. The purified anti-SIINFEKL-H2K^{b} IgG2a antibody was added at a final concentration of 3.5 μg/mL to induce ADCC, or not added to test “natural” cytotoxicity. Meanwhile, NK cells were enriched from 5 mouse spleens using an immunomagnetic negative selection kit from StemCell Technologies (Vancouver, Canada). The primary NK cells were added to the wells to achieve E:T ratios of 0.5:1, 1.5:1, and 5:1. In addition, monoculture wells were set up for both the targets (EL4 cells) and the effectors (NK cells). A phycoerythrin (PE) conjugated antibody against CD107a was included in every well. The plate was cultured for 4 hours at 37° C., and was then stained with SYTOX blue dead cell stain, Allophycocyanin (APC) conjugated Annexin V, and APC/Fire™ 750-conjugated anti-NKp46 antibody. Data was acquired on a Bio-Rad ZE5 flow cytometer (Univ of CT). The gating strategy used for analysis is depicted in FIG. 18.

On the basis of this gating strategy (FIG. 18), the flow cytometry-based outcomes of the experiments were generated, as illustrated in the Results section. In addition, samples of cell culture supernatants were collected at the end of the 4-hour coculture, and were assayed for lactate dehydrogenase (LDH) activity using a kit from Abcam (Cambridge, UK).

Key results: The anti-SIINFEKL-H2K^{b} antibody, with mouse isotype IgG2a, induces mouse NK cells to kill SIINFEKL-pulsed EL4 cells via the ADCC mechanism, as clearly established by the results summarized in FIG. 19. FIG. 19B illustrates that NK cytotoxic activity is increased in the presence of the antibody at all three E:T ratios. This boost in killing of target cells, which becomes increasingly significant with increasing E:T ratio, represents ADCC activity mediated by the added TCR-like antibody. At the very low E:T ratio of 0.5:1, ADCC contributed a 10.4% increase in target cell killing over the background “natural” cytotoxic activity of the NK cells when the antibody was absent. At the higher ratios of 1.5:1 and 5:1, the ADCC contribution was higher at 17.7% and 38.1%, respectively, and was statistically significant in both cases. Importantly, these coculture ADCC values were calculated after subtracting the direct cytotoxic effect of the antibody on the peptide-pulsed EL4 cells, illustrated in FIG. 19A.

In addition to the flow cytometry-based method of measuring cell death, a colorimetric enzyme-based assay was used to corroborate the results. Lactate dehydrogenase (LDH), a cytosolic enzyme that is only released into the culture medium when cells die, was quantified in supernatant samples from all monocultures and co-cultures at the end of the 4-hour incubation. In full agreement with the flow cytometry results, addition of the TCR-like antibody caused an increase in the cytotoxic activity of NK cells in a E:T ratio-dependent manner (FIG. 20). At the lowest ratio, LDH release trended upward without reaching significance. At the higher ratios of 1.5:1 and 5:1, the boost in cell death due to ADCC activity was 54.2% and 80%, respectively, and was statistically significant in both cases. In this assay, subtraction of the contribution of direct cytotoxic effects was not necessary, since less LDH was detected in the monoculture wells with antibody added.

FIG. 20 provides further corroboration (through LDH quantification) of the target cell death quantification observed through flow cytometry (FIG. 2). Additionally, the boost in antibody-induced ADCC cytotoxicity as a function of increasing E:T ratios observed through flow cytometry (FIG. 2) was re-confirmed by the LDH quantification (FIG. 20), which again confirmed the boost in antibody-induced ADCC cytotoxicity as a function of increasing E:T ratios.

FIG. 21 establishes that the ADCC quantification observed in FIG. 2 is a cumulative outcome of both apoptosis-based ADCC cytotoxicity (FIG. 21A) and necrosis-based ADCC cytotoxicity which is non-apoptotic (FIG. 21B). Additionally, the boost in antibody-induced ADCC cytotoxicity as a function of increasing E:T ratios, as observed in FIG. 19B, also holds true for both ADCC-apoptosis and ADCC-necrosis, where antibody-induced ADCC-apoptosis is boosted with increasing E:T ratios (FIG. 21A, boosted by 18.8%, 26.0%, and 49.8%, respectively) and antibody-induced ADCC-necrosis is also boosted with increasing E:T ratios (FIG. 21B, boosted by 20.9% for the highest ratio). As in FIG. 19, these values were calculated after subtracting the direct effect of the antibody on the peptide-pulsed EL4 cells, for each of these specific cell death mechanisms.

FIG. 22 clearly establishes that the degranulation process of NK cells, which is central to their innate immune function of targeting diseased cells, is enhanced during the antibody-based induction of the ADCC mechanism of NK cells (by a TCR-like antibody against a peptide-MHC complex), directed against target cells displaying the SIINFEKL-H2K^{b} complex. The boost in NK cell cytotoxic activity due to the presence of the antibody, by this metric, was highly significant at all three E:T ratios, with increases of 35.3, 39.2, and 18.2, respectively.

The experimental result represented by FIG. 23 establishes that the result obtained in the complementary experiment (represented by FIGS. 18-22) of ADCC induction is based on the specific binding of the SIINFEKL-H2K^{b} complex on the surface of the target EL4 cells by the anti-SIINFEKL-H2K^{b} antibody, and that this antibody binding is the basis of the ADCC quantification represented by FIGS. 19-21.

The observation of NK cell ADCC induction by the anti-SIINFEKL-H2K^{b} antibody binding to the SIINFEKL-H2K^{b} complex on the surface of the target EL4 cells was consistent between disparate methods of quantifying target cell death [SYTOX staining via flow cytometry (FIG. 19) and the LDH assay (FIG. 20)], and was also true for both of the underlying mechanisms (apoptosis and necrosis) for the ADCC-based cytotoxicity (FIG. 21).

Collectively, using SIINFEKL as a “surrogate neoepitope,” the experimental outcomes summarized in FIGS. 18-23 establish proof-of-concept that a TCR-like antibody against a neoepitope can induce the ADCC mechanism of NK cell cytotoxicity selectively against those target cells which display the neoepitope.

Discussion, Conclusions and Implications for NK cellular therapies: As outlined in FIGS. 19 and 20, significantly higher cytotoxicity was induced in the target cells by the effector NK cells in the presence of both antibody and effector cells, illustrating measurable manifestation of the ADCC (Antibody Dependent Cell-mediated Cytotoxicity) mechanism. Additionally, there is a dose-dependent increase in the ADCC killing of target cells, with higher Effector:Target (E:T) ratios resulting in higher ADCC killing. In other words, the data illustrates that antibodies can be generated against neoepitopes to induce the ADCC mechanism on NK cells for the purpose of NK cell killing of diseased cells displaying the neoepitope.

The biological significance of these experimental results is the experimental demonstration and establishment of the proof-of-concept or proof-of-principle of the following: a TCR-like antibody which has been specifically generated against a model neoantigen (i.e. the SIINFEKL-H2K^{b} complex) can induce the ADCC mechanism of Natural Killer (NK) effector cell cytotoxicity against the target cell displaying the neoantigen. This experimental demonstration illustrates an example of the advantages offered by the current invention in generating novel therapies for the treatment of cancer:

High specificity in targeting diseased cells such as cancer cells, while minimizing or avoiding toxicity towards healthy cells, by virtue of targeting neoepitopes which appear on the diseased cells and do not appear on healthy cells (de Sousa et al. (2021) Frontiers in Immunology, p. 1938).

Personalization of therapy, based on the generation of antibodies which specifically target neoepitopes that are manifested at the level of an individual patient or amongst a group of patients who share the neoepitope (Saini et al, (2017) Annals of Oncology, 28, pp. xii3-xii10).
Personalization is also facilitated at the level of each individual tumor, if neoepitope manifestation varies between different tumors within an individual patient.
Efficiency in the personalization of therapy, in terms of efficiency of time, or efficiency of resources to generate the therapy, or efficiency of treatment options or efficacy, as further elaborated below:
   Antibodies are the basis of the personalization in several embodiments of the current invention, rather than cellular therapy being the basis of personalization. Antibodies offer greater efficiencies in their generation and use compared to cellular therapy, for example they are faster and cheaper to produce.
   Antibodies may be administered to the patient as a single antibody (against a single neoepitope), or a combination of two or more antibodies (against two or more distinct epitopes), thus offering treatment efficiency.
   Exogenously added cellular therapy i.e. adoptive cellular therapy is optional and not a requirement, given that some embodiments of the current invention only require antibody administration to the patient, with a reliance on endogenous immune cells in the patient's body, and without any exogenous cellular therapy.
   Embodiments of the current invention offer efficiency in treatment. When exogenous cellular therapy is being administered in concert with the neoepitope targeting Antibody therapy, there is a range of cells to choose from (e.g. NK cells, macrophages, neutrophils etc.). Additionally, one or more types of cellular therapy may be administered for the purpose of synergy of biological effects, for example the administration of exogenous NK cells combined with exogenous macrophages for the induction of both the ADCC mechanism (through CD16 on NK cells) and the ADCP mechanism (through CD32 on macrophages) to fight the disease.
   Newer antibodies can continue to be generated in response to further mutations in the disease manifestation during the course of treatment (for example, in a scenario where different neoepitopes appear as the cancer metastasizes). This opportunity for treatment flexibility is offered by virtue of the resource-efficiency inherent to antibody generation versus cellular therapy generation. In other words, the therapy is adaptable to the disease.

In summary, the experimental results summarized in this section provide evidence that the current invention creates novel processes to generate personalized therapy by virtue of generating antibodies that selectively target neoepitopes on diseased cells, and the leveraging of the cellular mechanisms of ADCC, ADCP, or a combination of ADCC and ADCP.

### Planned Experiments

The experimental examples disclosed herein demonstrates that a TCR-like antibody specific for a particular peptide-MHC complex (in this case, a model antigen serving as a “surrogate neoepitope”) is capable of inducing ADCC activity by NK cells. Further experimentation is planned to [a] demonstrate that this ADCC activity leads to antitumor activity in vivo, [b] demonstrate that new TCR-like antibodies can be generated against known neoepitopes in humans using the methods described in the detailed description, and [c] demonstrate that the new anti-neoepitope TCR-like antibodies induce ADCC activity.

### Planned Experimental Strategy and Methodology:

Testing antitumor activity resulting from ADCC activity induced by the anti-SIINFEKL-H2K^{b} IgG2a antibody in vivo: Demonstrations of ADCC induction in vitro used the mouse tumor cell line EL4, when the EL4 cells were treated or “pulsed” with the SIINFEKL peptide (see Example 1). Another cell line, E.G7, is derived from EL4 but unlike EL4 cells, E.G7 is stably transfected with chicken egg ovalbumin (OVA), and given that OVA has the amino acid sequence SIINFEKL, E.G7 cells therefore present the “surrogate” neoepitope SIINFEKL on cell-surface H2K^{b} molecules even without separate exposure to the SIINFEKL peptide. Both of these tumor cell lines are tumorigenic in C57BL/6 mice, i.e. intradermal implantation leads to progressive tumor growth which can be monitored over time. In planned experiments using isotype-switched IgG2a version of the anti-SIINFEKL antibody (clone 25-D1.16), mice will be implanted with either [a] EL4 tumors, where EL4 cells have not been exposed to the SIINFEKL peptide (negative control), or [b] E.G7 tumors (which have the antibody's target on their surface), and treat the mice with various doses of the antibody. In some experiments, adoptive transfers of primary NK cells (freshly isolated from other C57BL/6 mice) will be concurrently performed to increase the number of NK cells available for ADCC-based antitumor activity, as a model of NK cell immunotherapy in humans. Endpoints will include the change in tumor size over time, and mouse survival.

Generation of new anti-neoepitope antibodies against a known “public” neoepitope in humans: Initial antibody development effort will focus on the HLA-A*02:01-binding neoepitope (amino acid sequence CLAVEEVSL) arising from a four-base-pair insertion in NPM1 that results in a brief alternative reading frame translation (Van der Lee, et al. (2019) J. Clin. Invest. 129(2):774-85). This frameshift mutation is found in 30% of AML patients, making it a “public” neoantigen. Also, HLA-A*02:01 is a highly prevalent HLA allele. The first phase of the project will have the following objectives: (1) Generate several novel TCR-like antibodies against this “public” frameshift-derived neoantigen commonly found in AML, and (2) test the candidate antibodies in vitro to evaluate their ability to induce ADCC activity by human NK cells against AIL cells. The top performing candidate antibodies (about 3-5 different antibodies) will constitute a shortlist to be tested preclinically in the second phase of the project.

To pursue the first objective, the steps highlighted in gray in FIG. 24 will be followed. In the first step, the “public” neoepitope peptide CLAVEEVSL will be synthesized. Importantly, it has been shown that the actual presented form of this peptide includes cysteinylation of the N-terminal cysteine residue (Van der Lee 2019). Therefore, the peptide synthesized for these experiments will need to be cysteinylated as well. Following synthesis of peptide, HLA-peptide complexes using recombinant HLA-A*02:01 and beta-2-microglobulin (β2m) proteins will be produced. Monomeric complexes, without biotinylation or fluorophore conjugation, will be produced for the purpose of immunizing animals and performing ELISAs (discussed below); in addition, fluorophore-conjugated HLA-peptide tetramers will be produced for the purpose of fluorescence-activated cell sorting.

A protocol for the rapid generation of high-affinity antibodies specific for HLA-peptide complexes (i.e. “TCR-like” antibodies) was published in 2017 (Ouisse, et al. (2017) BMC Biotechnol. 17(1):3). This protocol utilizes OmniRats, which are rats genetically engineered with human antibody genes in place of their natural rat antibody genes. When these rats are immunized with an antigen, the resulting antibodies have fully human sequences. OmniRats will be immunized twice with HLA-A*02:01-CLAVEEVSL complexes (unconjugated monomers), with the immunizations spaced 16 days apart. Five days after the second immunization, spleens and lymph nodes will be harvested and dissociated into single-cell suspensions.

Immunomagnetic negative selection kits will be used to enrich the B cells by removing most T cells and other cells present in the dissociated lymphoid tissues. The resulting cell suspensions, consisting of greater than 90% B cells, will be prepared for the next step, fluorescence-activated cell sorting. The purpose of this step will be to isolate individual B cells whose B cell receptors specifically bind the HLA-A*02:01-CLAVEEVSL complex. Cells will be stained with antibodies against rat IgG (to select B cells) and the T cell receptor (to exclude T cells), and several fluorescently labeled HLA-peptide tetramer complexes. As shown in Ref #, B cells with specificity for a particular HLA-peptide complex are successfully isolated using the following staining and gating strategy: (1) Cells are stained with tetramers of the HLA-peptide of interest, but two different fluorophores are used. For example, cells are stained with tetramer conjugated to Fluorescein isothiocyanate (FITC) and simultaneously stained with the same tetramer conjugated to Phycoerythrin (PE). Meanwhile, the cells are also stained with another tetramer of an irrelevant HLA-peptide complex conjugated to a different fluorophore (e.g. APC). (2) During cell sorting, a gate is set to select those B cells positive for both of the fluorophores associated with the tetramer of interest (in this case FITC and PE), i.e. double-positive cells. For those double-positive cells, an additional gate is set to select those cells negative for the fluorophore associated with the irrelevant tetramer (in this case APC). Using two different fluorophores for positive selection of the tetramer of interest excludes B cells specific for the fluorophores; meanwhile, the negative gate on the irrelevant tetramer excludes B cells specific for the HLA molecule itself (rather than the antigen peptide in association with the HLA molecule) and also excludes B cells specific for β2m. The resulting number of individual B cells collected is small (less than 100), but some of them show high specificity and high affinity for the HLA-peptide complex used for immunization (˜12%).

Individual single B cells will be sorted into separate PCR tubes. Following reverse transcription into cDNA and PCR-based amplification of the human heavy and light chain genes, DNA sequencing will be performed to obtain the unique sequences of the variable regions. The variable region sequences will be cloned into a eukaryotic expression vector encoding a human antibody of the IgG1 isotype. CHO cells will be transiently transfected to produce the anti-HLA-neoepitope antibodies. Supernatants will be collected from transfected CHO cell cultures, and antibody purification will be performed using human IgG isolation kits. It is expected that 20-80 B cell clones will be isolated from a single immunized OmniRat; therefore, it is expected that 2-9 fully human CLAVEEVSL-specific candidate antibodies will result from following this process using a single animal. This process will be followed with however many OmniRat immunizations are necessary to produce 30-50 candidate antibodies to be tested for antigen specificity.

In order to test the specificity of the candidate antibodies, the same HLA-A*02:01-CLAVEEVSL complexes (monomers) will be used that were used to immunize the animals, Using standard ELISA plates, wells will be coated with these HLA-peptide complexes or irrelevant HLA-peptide complexes. Each antibody will be individually tested for binding to these HLA-peptide complexes. Those which show only weak binding to HLA-A*02:01-CLAVEEVSL, or show binding significantly greater than background to the irrelevant complex, will be discarded. Those which bind strongly to HLA-A*02:01-CLAVEEVSL, but not to the irrelevant HLA-peptide complex, will be kept as candidate antibodies to be tested further in ADCC assays (the second objective).

### Testing ADCC Activity of New Anti-Neoepitope TCR-Like Antibodies:

To pursue the second objective, experiments will be performed in vitro that test the ability of these anti-neoepitope “TCR-like” candidate antibodies to induce the ADCC mechanism of NK cell cytotoxicity. Preliminary experimentation has demonstrated that a TCR-like antibody specific for the SIINFEKL-H2K^{b} complex (a model antigen serving as a “surrogate neoepitope”) is capable of inducing ADCC activity by NK cells.

Testing of candidate anti-HLA-A*02:01-CLAVEEVSL antibodies will follow an experimental design similar to that of the preliminary experiments disclosed herein. However, rather than using cell lines as target cells, cancer cells sourced from AML patients will be used. These cancer cells will be procured from a university teaching hospital, e.g. UConn Health or University of Minnesota. Since candidate antibodies will be specific for a neoepitope presented by HLA-A*02:01, most of the AML samples will be obtained from patients possessing that HLA allele. However, a small number of AML samples lacking that allele will also be obtained to serve as negative controls. AML samples will undergo Sanger sequencing to test for the presence of the particular four-base-pair insertion in NPM1, which ought to be present in 30% of the samples, that leads to the presentation of the “public” neoepitope CLAVEEVSL on HLA-A*02:01. All samples positive for HLA-A*02:01, and also possessing the correct mutation, will be selected for use in ADCC experiments. However, some HLA-A*02:01-positive samples lacking the correct mutation to serve as negative controls will also be selected.

For effector cells in the ADCC assays, primary human NK cells from healthy donors will be used. Fresh buffy coats will be procured from Research Blood Components (Watertown, MA), and the PBMCs will be obtained using density gradient centrifugation. NK cells will be enriched using immunomagnetic negative selection.

Each of the candidate antibodies will be tested for their ability to induce ADCC by NK cells against AML cells with or without the HLA-A*02:01 allele, and with or without the frameshift mutation that results in presentation of CLAVEEVSL. Specifically, each experiment will include at least four AML samples possessing the correct allele and correct mutation, at least one AML sample lacking the correct allele, and at least one AML sample lacking the correct mutation. For each antibody, cocultures will be set up at multiple effector:target ratios (e.g. 1:1, 4:1, and 10:1) and at multiple antibody concentrations (e.g. 0 μg/mL, 0.02 μg/mL, 0.2 μg/mL, 2 μg/mL). Isotype control antibodies will also be included as a negative control. Cocultures will proceed for 4-6 hours. Cultures of AML cells in the absence of effector (NK) cells will be treated similarly, to establish the baseline death of these AML cells under these conditions. Death of AML cells will be quantified using flow cytometry, following staining with a membrane impermeable nuclear stain (e.g. propidium iodide) as well as an apoptosis marker (e.g. Annexin V). There will be four technical replicates for each group.

Some amount of cytotoxic activity toward the target AML cells, even in the absence of added antibody, is expected to be observed in these assays. This is due to the non-antigen-specific mechanisms of NK cell recognition and killing. The key readout when testing ADCC is that NK cell-mediated killing is enhanced above this baseline in the presence of antibody. Increased killing (ADCC activity) is expected in the presence of antibody only when (1) the AML sample contains the NPM1 frameshift mutation leading to presentation of the “public” neoepitope CLAVEEVSL, and (2) the AML sample expresses HLA-A*02:01. The 3-5 antibodies inducing the greatest ADCC activity will be chosen for future animal experimentation.

### ENUMERATED EMBODIMENTS

The following enumerated embodiments are provided, the numbering of which is not to be construed as designating levels of importance.

Embodiment 1 provides a composition for treating cancer, said composition comprising:
a. one or more antibodies, or antigen-binding fragments thereof that specifically bind one or more tumor-specific epitopes, and
b. an effective amount of a cytotoxic immune effector cell.

Embodiment 2 provides the composition of embodiment 1, wherein the composition is personalized.

Embodiment 3 provides the composition of embodiment 1, wherein the tumor-specific epitope is a neoepitope.

Embodiment 4 provides the composition of embodiment 1, wherein the antibodies or antigen-binding fragments thereof are humanized.

Embodiment 5 provides the composition of embodiment 1, wherein the antibodies or antigen-binding fragments thereof are fully human.

Embodiment 6 provides the composition of embodiment 1, wherein the antibodies or antigen-binding fragments thereof are afucosylated.

Embodiment 7 provides the composition of embodiment 1, wherein the antibody or antigen-binding fragment thereof is bispecific.

Embodiment 8 provides the composition of embodiment 1, wherein the tumor-specific epitope is a neoepitope/HLA complex.

Embodiment 9 provides the composition of embodiment 1, wherein the cytotoxic immune effector cells are a cell type selected from the group consisting of NK cells, macrophages, monocytes, neutrophils, dendritic cells, or any combination thereof.

Embodiment 10 provides the composition of embodiment 1, wherein the cytotoxic immune effector cells are irradiated.

Embodiment 11 provides a method of producing a composition for treating cancer, said method comprising:
a. obtaining a tumor specimen from a subject and identifying tumor-specific genetic mutations expressed by the tumor through sequencing,
b. generating HLA/neoepitope complexes based on the identified tumor-specific genetic mutations,
c. immunizing a non-human animal with the HLA/neoepitope complexes,
d. isolating B cells from the immunized non-human animal that produce HLA/neoepitope-specific antibodies or facilitate the generation of antigen-binding fragments thereof,
e. verifying that the HLA/neoepitope-specific antibodies bind to the tumor,
f. producing an effective amount of the HLA/neoepitope-specific antibodies, and
g. combining the effective amount of the HLA/neoepitope-specific antibodies with an effective amount of a cytotoxic immune effector cell.

Embodiment 12 provides the method of embodiment 11, wherein the sequencing is next-generation sequencing.

Embodiment 13 provides the method of embodiment 11, wherein the non-human animal is humanized.

Embodiment 14 provides the method of embodiment 11, wherein the non-human animal produces fully-human antibodies.

Embodiment 15 provides the method of embodiment 13, wherein the non-human humanized animal is selected from the group consisting of a rat, a mouse, a rabbit, a donkey, a goat, a camel, a llama, an alpaca, and a shark.

Embodiment 16 provides the composition of embodiment 11, wherein the HLA/neoepitope-specific antibodies are generated through a non-animal method.

Embodiment 17 provides the composition of embodiment 16, wherein the non-animal method of phage display.

Embodiment 18 provides the composition of embodiment 11, wherein the antibodies or antigen-binding fragments thereof are afucosylated.

Embodiment 19 provides the composition of embodiment 11, wherein the isotype of the antibodies supports ADCC or ADCP.

Embodiment 20 provides the composition of embodiment 11, wherein the isotype of the antibodies are further modified from an isotype that does not support ADCC or ADCP to an isotype that supports ADCC or ADCP.

Embodiment 21 provides the method of embodiment 11, wherein the HLA/neoepitope complexes are selected from the group consisting of monomeric complexes, pentameric complexes, tetrameric complexes, dextran-linked complexes, and any combination thereof.

Embodiment 22 provides the method of embodiment 11, wherein the cytotoxic immune effector cells are a cell type selected from the group consisting of NK cells, macrophages, monocytes, neutrophils, dendritic cells, or any combination thereof.

Embodiment 23 provides a method of producing a composition for treating cancer, said method comprising:
a. obtaining a tumor specimen from a subject and identifying tumor-specific genetic mutations expressed by the tumor through sequencing,
b. generating HLA/neoepitope complexes based on the identified tumor-specific genetic mutations,
c. immunizing a non-human animal with the HLA/neoepitope complexes,
d. isolating B cells from the immunized non-human animal that produce HLA/neoepitope-specific antibodies or facilitate the generation of antigen-binding fragments thereof,
e. verifying that the HLA/neoepitope-specific antibodies bind to the tumor,
f. generating one or more a chimeric antigen receptors (CARs) comprising the antigen binding domains from the HLA/neoepitope-specific antibodies, and
g. expressing the CARs in a cytotoxic immune effector cell.

Embodiment 24 provides the method of embodiment 23, wherein the sequencing is next-generation sequencing.

Embodiment 25 provides the method of embodiment 23, wherein the non-human animal is humanized.

Embodiment 26 provides the method of embodiment 23, wherein the non-human animal produces fully-human antibodies.

Embodiment 27 provides the method of embodiment 23, wherein the non-human humanized animal is selected from the group consisting of a rat, a mouse, a rabbit, a donkey, a goat, a camel, a llama, an alpaca, and a shark.

Embodiment 28 provides the method of embodiment 23, wherein the HLA/neoepitope-specific antibodies are generated through a non-animal method.

Embodiment 29 provides the method of embodiment 23, wherein the non-animal method is phage display.

Embodiment 30 provides the composition of embodiment 23, wherein generating the CAR further comprises the step of generating an scFv based on the antigen-binding domains of the antibodies.

Embodiment 31 provides the composition of embodiment 23, wherein the CAR further comprises a transmembrane domain, a signaling domain, and a costimulatory domain.

Embodiment 32 provides the method of embodiment 23, wherein the HLA/neoepitope complexes are selected from the group consisting of monomeric complexes, pentameric complexes, tetrameric complexes, dextran-linked complexes, and any combination thereof.

Embodiment 33 provides the method of embodiment 23, wherein the cytotoxic immune effector cells are a cell type selected from the group consisting of NK cells, T cells, NK T cells, or any combination thereof.

Embodiment 34 provides the method of embodiment 23, wherein the cytotoxic immune effector cell is an NK cell.

Embodiment 35 provides the method of embodiment 23, wherein the cytotoxic immune effector cell is a CD8+ T cell.

Embodiment 36 provides a method of treating cancer in a subject in need thereof, said method comprising:
a. obtaining a tumor specimen from the subject and identifying tumor-specific neoepitopes expressed by the tumor through sequencing,
b. generating HLA/neoepitope complexes from the identified tumor-specific neoepitopes,
c. immunizing a non-human mammal with the HLA/neoepitope complexes,
d. isolating B cells that produce HLA/neoepitope-specific antibodies or antigen-binding fragments thereof from the immunized non-human mammal,
e. verifying that the HLA/neoepitope-specific antibodies bind to the tumor,
f. producing an effective amount of the HLA/neoepitope-specific antibodies, and
g. administering an effective amount of the HLA/neoepitope-specific antibodies and an effective amount of a cytotoxic immune effector cell to the subject, thereby treating the cancer.

Embodiment 37 provides the method of embodiment 36, wherein the sequencing is next-generation sequencing.

Embodiment 38 provides the method of embodiment 36, wherein the non-human animal is humanized.

Embodiment 39 provides the method of embodiment 36, wherein the non-human animal produces fully-human antibodies.

Embodiment 40 provides the method of embodiment 36, wherein the non-human humanized animal is selected the group consisting of a rat, a mouse, a rabbit, a donkey, a goat, a camel, a llama, an alpaca, and a shark.

Embodiment 41 provides the method of embodiment 36, wherein the HLA/neoepitope-specific antibodies are generated through a non-animal method.

Embodiment 42 provides the method of embodiment 36, wherein the non-animal method is phage display.

Embodiment 43 provides the method of embodiment 36, wherein the HLA/neoepitope-specific antibodies are afucosylated.

Embodiment 44 provides the method of embodiment 36, wherein the isotype of the antibodies supports ADCC or ADCP.

Embodiment 45 provides the method of embodiment 36, wherein the isotype of the antibodies are further modified from an isotype that does not support ADCC or ADCP to an isotype that supports ADCC or ADCP.

Embodiment 46 provides the method of embodiment 36, wherein the HLA/neoepitope complexes are selected from the group consisting of monomeric complexes, pentameric complexes, tetrameric complexes, dextran-linked complexes, and any combination thereof.

Embodiment 47 provides the method of embodiment 36, wherein the cytotoxic immune effector cells are a cell type selected from the group consisting of NK cells, macrophages, monocytes, neutrophils, dendritic cells, or any combination thereof.

Embodiment 48 provides a method of treating cancer in a subject in need thereof, said method comprising:
a. obtaining a tumor specimen from the subject and identifying tumor-specific neoepitopes expressed by the tumor through sequencing,
b. generating HLA/neoepitope complexes from the identified tumor-specific neoepitopes,
c. immunizing a non-human mammal with the HLA/neoepitope complexes,
d. isolating B cells that produce HLA/neoepitope-specific antibodies or antigen-binding fragments thereof from the immunized non-human mammal,
e. verifying that the HLA/neoepitope-specific antibodies bind to the tumor,
f. generating a chimeric antigen receptor (CAR) comprising the antigen-binding domains from the HLA/neoepitope-specific antibodies,
g. expressing the CAR in a cytotoxic immune effector cell, and
h. administering to the subject an effective amount of the cytotoxic immune effector cell, thereby treating the cancer.

Embodiment 49 provides the method of embodiment 48, wherein the sequencing is next-generation sequencing.

Embodiment 50 provides the method of embodiment 48, wherein the non-human animal is humanized.

Embodiment 51 provides the method of embodiment 48, wherein the non-human animal produces fully-human antibodies.

Embodiment 52 provides the method of embodiment 48, wherein the non-human humanized animal is selected from the group consisting of a rat, a mouse, a rabbit, a donkey, a goat, a camel, a llama, an alpaca, and a shark.

Embodiment 53 provides the method of embodiment 48, wherein the HLA/neoepitope-specific antibodies are generated through a non-animal method.

Embodiment 54 provides the method of embodiment 48, wherein the non-animal method is phage display.

Embodiment 55 provides the method of embodiment 48, wherein the HLA/neoepitope complexes are selected from the group consisting of monomeric complexes, pentameric complexes, tetrameric complexes, dextran-linked complexes, and any combination thereof.

Embodiment 56 provides the composition of embodiment 48, wherein generating the CAR further comprises the step of generating an scFv based on the antigen-binding domains of the antibodies.

Embodiment 57 provides the composition of embodiment 48, wherein the CAR further comprises a transmembrane domain, a signaling domain, and a costimulatory domain.

Embodiment 58 provides the method of embodiment 48, wherein the cytotoxic immune effector cells are a cell type selected from the group consisting of NK cells, T cells, NK T cells, or any combination thereof.

Embodiment 59 provides the method of embodiment 48, wherein the cytotoxic immune effector cell is an NK cell.

Embodiment 60 provides the method of embodiment 48, wherein the cytotoxic immune effector cell is a CD8+ T cell.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention 5 has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-34. (canceled)

35. A composition for treating cancer, said composition comprising:
a. two or more antibodies or antigen-binding fragments thereof that specifically bind one or more tumor-specific epitopes, and
b. an effective amount of two or more cytotoxic immune effector cells.

36. The composition of claim 35, wherein:
a. at least one of the antibodies or antigen-binding fragments thereof can engage with Fc-gamma receptors on an effector cell; and
b. at least one of the antibodies or the antigen binding fragments thereof is bispecific and binds to an immune cell epitope.

37. The composition of claim 36, wherein:
a. the Fc-gamma receptor expressing effector cell is selected from the group consisting of a NK cell, macrophage, monocyte, neutrophil, dendritic cell, gamma-delta T cell, and any combination thereof; and
b. the immune cell epitope is expressed by an immune cell selected from the group consisting of T cells, B cells, and any combination thereof.

38. The composition of claim 35, wherein the tumor-specific epitope is a neoepitope.

39. The composition of claim 35, wherein the composition in personalized.

40. The composition of claim 35, wherein the antibodies or antigen-binding fragments thereof are humanized.

41. The composition of claim 35, wherein the antibodies or antigen-binding fragments thereof are fully human.

42. The composition of claim 35, wherein the antibodies or antigen-binding fragments thereof are afucosylated.

43. The composition of claim 35, wherein the tumor-specific epitope is a neoepitope/HLA complex.

44. The composition of claim 35, wherein:
a. at least one of the cytotoxic immune effector cells is an Fc-gamma receptor expressing effector cell selected from the group consisting of NK cells, macrophages, monocytes, neutrophils, dendritic cells, gamma-delta T cells and any combination thereof, and
b. at least one of the cytotoxic immune effector cells is selected from the group consisting of T cell, B cells, and any combination thereof.

45. The composition of claim 35, wherein the immune effector cells are irradiated.

46. A composition for treating cancer, said composition comprising:
a. an effective amount of one or more antibodies, or antigen-binding fragments thereof that specifically bind one or more tumor-specific epitopes, and
b. an effective amount of one or more cytotoxic immune effector cells.

47. The composition of claim 46, wherein the tumor-specific epitope is a neoepitope.

48. The composition of claim 46, wherein the composition is personalized.

49. The composition of claim 46, wherein the antibodies or antigen-binding fragments thereof are humanized.

50. The composition of claim 46, wherein the antibodies or antigen-binding fragments thereof are fully human.

51. The composition of claim 46, wherein the antibodies or antigen-binding fragments thereof are afucosylated.

52. The composition of claim 46, wherein the antibody or antigen-binding fragment thereof is bispecific.

53. The composition of claim 46, wherein the tumor-specific epitope is a neoepitope/HLA complex.

54. The composition of claim 46, wherein the cytotoxic immune effector cells are Fc-gamma receptor expressing effector cells selected from the group consisting of NK cells, macrophages, monocytes, neutrophils, dendritic cells, gamma-delta T cells, and any combination thereof.

55. The composition of claim 46, wherein the cytotoxic immune effector cells are irradiated.
